SEQUENCE LISTING (45 sequences)

SEQ ID NO: 1 - DNA, 966 bp, synthetic construct
  source 1..966: mol_type = other DNA
atgtcaaccacaaaagaagaccaacctctccccactccccccttccacatcgtccccaacatcaacaaccttcgagacgccgccctcttccccctaaccaccccctccggctcaatccgccccaaaatcctcttccgctccgccgacgtctccaaactccccctcagcggctggcaagccctccattccctgggtatcacccatgtcttcgacctacgcagcgcgcccgaagtcggattccgcgactcggacacctccaagccagaatgggtcagcgccatgacctccgtcggcatcaagcgcacgtggtgccccgtcttcactgaagcagactactcccccgagggcctcgccaagcgctacgtcaagtacatggacgaagacgtggcgggctttgtatccgcgtaccacgacatccttctcgacggcggcgcagcatacaccactatcctgcgccacctaattgatcatcccggcgagggagtgttgattcactgcacggcgggcaaagaccgcacgggcatcttcttcggcttgctattcgcctacctgggcgtggacagccaagtcattgctgaggagtacaacctgacggaactcgggctgacgcatgtgcgcgaagaagtcgtcgcgagactgttgcagtccccggcgtttaagaattacatcgcgaccaaggcgaccggaaagcagcttagcgccgaggagattgggaagttgattgcggacgacaaggcggggaagcagagtggtgtcgaggagacattggatccggaggcgaggaagcagggcagggaggctgcgctgaggatggtgggggcgaagaaggagacgatggttaaggcgttggagatgctggagagggattttgggggcgcggagaagtatctgcgcgagaagtgtggtctgggagatggggatttggagaagttgaggaggaatttggtggtgcgtgaggaggcgtga

SEQ ID NO: 2 - DNA, 966 bp, synthetic construct
  source 1..966: mol_type = other DNA
atgtctacaactaaagaagatcaaccattgcccacgcctccattccacatcgttccaaacattaacaacttgagagacgctgccttattcccattgaccactccatctggttccatcagaccaaagattttgttcagatctgctgacgtttccaaattacctttgtctggttggcaagccttgcactctttgggtattactcacgtcttcgatttaagatctgccccagaagtcggttttagagattctgatacctccaagccagaatgggtttctgctatgacctccgttggtatcaagagaacctggtgtcctgttttcactgaagccgattactctcctgaaggtttggctaagcgttacgtcaagtacatggatgaggatgttgctggtttcgtttctgcttatcatgacattttgttggacggtggtgctgcttatactaccattttgagacacttaatcgatcatccaggtgaaggtgtcttgattcactgtactgctggtaaggatcgtactggtatcttcttcggtttgttgttcgcttacttgggtgttgattcccaagttattgccgaagaatataacttgactgaattgggtttgactcacgttagagaagaagttgtcgctagattgttgcaatccccagctttcaagaactacattgccactaaggctaccggtaaacaattgtctgctgaagaaatcggtaagttgatcgctgacgataaggctggtaaacaatccggtgttgaagaaactttggatccagaggctagaaaacaaggtagagaagctgctttgagaatggttggtgctaaaaaagaaactatggttaaggctttggagatgttggaaagagattttggtggtgctgaaaagtacttgagagaaaaatgtggtttgggtgacggtgacttggaaaagttgcgtagaaacttggttgtcagagaagaagcttaa

SEQ ID NO: 3 - protein, 321 aa, synthetic construct
MSTTKEDQPLPTPPFHIVPNINNLRDAALFPLTTPSGSIRPKILFRSADVSKLPLSGWQALHSLGITHVFDLRSAPEVGFRDSDTSKPEWVSAMTSVGIKRTWCPVFTEADYSPEGLAKRYVKYMDEDVAGFVSAYHDILLDGGAAYTTILRHLIDHPGEGVLIHCTAGKDRTGIFFGLLFAYLGVDSQVIAEEYNLTELGLTHVREEVVARLLQSPAFKNYIATKATGKQLSAEEIGKLIADDKAGKQSGVEETLDPEARKQGREAALRMVGAKKETMVKALEMLERDFGGAEKYLREKCGLGDGDLEKLRRNLVVREEA

SEQ ID NO: 4 - DNA, 873 bp, synthetic construct
  source 1..873: mol_type = other DNA
atgagtctttctcttccttttattcatgtggagggggtgagcaactttaggagcttgggtggatacccagtcgccacatgtacacctggcaaaaaaccacttaccacccgtcagcatttcttctaccgcagtgcggatctggtcaagataacggaatccggccgagctacgatgcaatcgctgggggttacaagcgtgtatgatctgcgctccgttggcgaaggtcaaagggcacaggccgcacacaaccaggcaggcatcaccacaagattcaatagcatagctctagggcccgacatcaaggtgcacccaactcccatttttgcccacgaggactggagcccggaagcagtaggcgagcggtaccttcggtatgcccaggagcacgccctatccggctcgggctatgcagaggtctaccgagacatgctagaacggggacacgaggccatccgcgtcgtccttctgcacgtacgagaccatccgaccgagccctttctctgtcattgtagcgcaggcaaggaccgcacgggtgtcgtcgtcgccgtgttgctaaagctggccggatgcgacgatgaggtggtggcacgcgaatatgcgctcacggagctgggactggcggcccgtaaggagtttatcgtgcagtacctcttgcggaaaccagaggtgaaaggttcacgggtgctcgccgagcgagtagccagtgcgtcttattccaacatgtgggagaccctgcagatggtccgaagcaaatacgggagcatggctggttatgtgatgaagttctgtagtcttaccgagtctgatctggacagcatccaacagaatctgacctgctcggatgcgccgagtccgtgggccgtatcgatatag

SEQ ID NO: 5 - DNA, 873 bp, synthetic construct
  source 1..873: mol_type = other DNA
atgagcctttccctgccttttattcatgtcgagggtgtctctaatttcagatccttaggtggttaccctgttgccacttgtactccaggtaagaagccattaactaccagacaacacttcttctacagatctgctgatttagtcaagattactgaatctggtagagctaccatgcaatctttgggtgttacttctgtttacgacttgagatccgtcggtgaaggtcaacgtgcccaagctgctcataaccaagccggtattactaccagattcaattccattgctttaggtccagacattaaggttcaccctactccaattttcgctcatgaagattggtcccctgaagctgttggtgaaagatatttaagatacgctcaagaacatgctttgtccggttctggttacgctgaagtttacagagatatgttggaaagaggtcatgaagccatcagagttgttttgttgcacgtcagagatcacccaaccgaaccatttttgtgtcattgttctgctggtaaggatagaaccggtgttgtcgtcgccgttttgttgaagttagctggttgtgatgacgaagttgttgctagagaatatgctttaactgaattgggtttggctgctagaaaggaattcattgttcaatacttgttgagaaagcctgaagtcaaaggttctagagtcttggccgaaagagttgcctccgcctcctactctaacatgtgggaaactttgcaaatggttagatctaaatacggttctatggctggttacgttatgaagttctgttctttgactgaatctgatttggactctattcaacaaaacttgacctgttccgatgctccatctccttgggctgtctccatttag

SEQ ID NO: 6 - protein, 290 aa, synthetic construct
MSLSLPFIHVEGVSNFRSLGGYPVATCTPGKKPLTTRQHFFYRSADLVKITESGRATMQSLGVTSVYDLRSVGEGQRAQAAHNQAGITTRFNSIALGPDIKVHPTPIFAHEDWSPEAVGERYLRYAQEHALSGSGYAEVYRDMLERGHEAIRVVLLHVRDHPTEPFLCHCSAGKDRTGVVVAVLLKLAGCDDEVVAREYALTELGLAARKEFIVQYLLRKPEVKGSRVLAERVASASYSNMWETLQMVRSKYGSMAGYVMKFCSLTESDLDSIQQNLTCSDAPSPWAVSI

SEQ ID NO: 7 - DNA, 957 bp, synthetic construct
  source 1..957: mol_type = other DNA
atgtcgagcccccaagccctcccctccccgcccttccacaacatccccaacattgccaacctccgagacgccgctctcttccccctccagacggccacaggaccactcaaaccctccctcctcttccgctccgccgacgtctccaaattgcttcccgagaactggtcatccctctccgccctcggcgtcacgcacgtgttcgacctgcgcagcgccccagaagtcggcttcacctcatcgaccccttccacctcgctgcctacctgggtatcgtccatgaacgaagcaggcatcaaccggacctgggtccccgtcttcgcggaacaagactactcgcccgaaggcctggcgaagcgatatgtaaagtacatggacgagtcggtcacgggcttcgtctctgcttaccacgacatcctgctcgctgcgggccctgcctatcgctccatcctactgtacctgatccgctccccgggcgccggtgtattggtgcactgcaccgccggcaaagaccggacgggcatcttcttcggcatcgtattcgactacctgggcgtcgatcggcaggccattgccgacgagtacaatctgaccgaacagggactggggagcgtgcgggaggaggtcgtagcgaggttgatgaagagccctgcgttccgcaactatatgaacacgaagcagacgggcgagcagcttagtaccgaggagattgggcgcttgattcaagaagagaaggagggtaaggctccggctgctgaagaagagctcgacccagagacgcgcgaggtggggcgtcaggctgcgttgaggatggtgggcgcacgcaaggagacgatgattgcggcgctgcagatggtggataaggtgtttgggggctcggagaagtatctgagggagtattgtggacttggggataaggatctcgaggcgctgaggaggaacctggtggatggggcatga

SEQ ID NO: 8 - DNA, 957 bp, synthetic construct
  source 1..957: mol_type = other DNA
atgtccagtccacaggcgcttcctagtccacccttccacaacattccaaacatcgctaatttgagagacgccgctttattcccattacaaactgctactggtccattgaagccatccttattgttccgttctgccgatgtctctaaattgttaccagaaaactggtcctctttgtctgctttgggtgtcactcacgttttcgatttgagatctgccccagaagttggtttcacttcttctaccccttctacctctttgccaacttgggtttcttccatgaacgaagctggtatcaacagaacttgggtccctgtttttgccgaacaagactactctccagaaggtttggctaagagatacgtcaagtacatggatgaatctgtcaccggtttcgtctctgcctatcatgatattttgttggctgctggtcctgcttacagatctatcttgttgtacttgatcagatccccaggtgccggtgttttagttcactgtactgccggtaaagatagaactggtattttcttcggtattgtcttcgattacttgggtgtcgatcgtcaagccattgccgacgaatacaacttgactgaacaaggtttgggttctgtcagagaagaagtcgttgccagattgatgaaatcccctgctttcagaaactacatgaacactaagcaaactggtgaacaattgtctactgaagaaatcggtagattgattcaagaagaaaaggaaggtaaagctccagccgccgaagaagaattggacccagaaaccagagaagttggtcgtcaagctgctttgagaatggttggtgctagaaaagaaaccatgatcgccgccttgcaaatggtcgacaaggtttttggtggttctgaaaagtacttaagagaatactgtggtttgggtgataaggacttggaagctttaagaagaaacttagttgatggtgcctaa

SEQ ID NO: 9 - protein, 318 aa, synthetic construct
MSSPQALPSPPFHNIPNIANLRDAALFPLQTATGPLKPSLLFRSADVSKLLPENWSSLSALGVTHVFDLRSAPEVGFTSSTPSTSLPTWVSSMNEAGINRTWVPVFAEQDYSPEGLAKRYVKYMDESVTGFVSAYHDILLAAGPAYRSILLYLIRSPGAGVLVHCTAGKDRTGIFFGIVFDYLGVDRQAIADEYNLTEQGLGSVREEVVARLMKSPAFRNYMNTKQTGEQLSTEEIGRLIQEEKEGKAPAAEEELDPETREVGRQAALRMVGARKETMIAALQMVDKVFGGSEKYLREYCGLGDKDLEALRRNLVDGA

SEQ ID NO: 10 - DNA, 930 bp, synthetic construct
  source 1..930: mol_type = other DNA
atgtctgatgacacctttcccacggctgctcccgggacagtaccttcttcgcggtttctttccgtgggcggagtagtcaatttccgtgaactgggcggttacccttgcagtgctctccctcctgcctcaaacggctcaccggacaacgcgtcggattcgacacctcggggtagccactcgtgcatccggcctggatttctctttcgatcggctcagccgtcccaggttaccccagccggtatcgagacactagtacacgaacttcgcatccgggcgatttttgactttcgctcacagaccgaaattcagcttgtcaccactcgctatcctgattcgctactcgagatacctggtacgactcgctattccgtgccggtcttctcggagggtgattattccccggcgtcattagtcaagaagtacggagtgtcctccgatactgccgtggattccatttcctccacaaacgccaagcctacaggattcgtccacgcgtatgaggccatcgcacgcagtgctgcaggaaacggtagttttcgtaagataacggaccacataatacaacatccggaccggccaatcctatttcactgcacactgggaaaagaccgaaccggtgtatttgcagcattgctattgagtctttgcggggtaccaaacgagactatagttgaagactatgctatgactactgagggcttcggggcctggcgagaacacctagtccaacgcttgttgcaaagaaaggatgcagctacacgtgaagaggcagaatccattattgccagccacccggagactatgaaggcttttcttgaagatgtggtagcggcgaagtttgggagtgctcgaaattattttgtccagcaatgtggattaacgcaagcagacgttgataaattaattcatacattggtctttacgaattga

SEQ ID NO: 11 - DNA, 930 bp, synthetic construct
  source 1..930: mol_type = other DNA
atgtccgatgacacattcccgaccgctgctcctggtactgtcccatcctccagattcttgtccgtcggtggtgtcgtcaacttcagagaattaggtggttacccttgttctgctttgccaccagcctccaatggttcccctgacaacgcttctgactctacccctagaggttcccattcctgtatcagaccaggtttcttattccgttctgctcaaccatcccaagtcactccagctggtatcgaaaccttggtccatgaattgcgtatccgtgccatttttgactttagatctcaaaccgagattcaattggtcactactagatacccagattccttgttagaaattccaggtactactagatactctgtcccagtcttctctgaaggtgactactctccagcttctttggtcaagaaatacggtgtttcttctgacactgctgttgattctatttcctctaccaatgctaaacctaccggtttcgttcacgcttacgaagccattgccagatccgctgctggtaatggttcttttagaaagattaccgaccacattattcaacaccctgatagacctattttgtttcactgcactttaggtaaggatagaaccggtgtcttcgccgctttattgttgtctttgtgtggtgtccctaatgaaactattgttgaagactacgccatgactactgaaggtttcggtgcttggagagaacacttggttcaaagattgttgcaaagaaaagacgctgctactagagaagaagctgaatctatcatcgcctctcatccagaaactatgaaagctttcttagaggatgttgttgctgctaagttcggttccgctagaaactacttcgtccaacaatgtggtttgactcaagctgacgttgacaagttgatccatactttggttttcaccaactaa

SEQ ID NO: 12 - protein, 309 aa, synthetic construct
MSDDTFPTAAPGTVPSSRFLSVGGVVNFRELGGYPCSALPPASNGSPDNASDSTPRGSHSCIRPGFLFRSAQPSQVTPAGIETLVHELRIRAIFDFRSQTEIQLVTTRYPDSLLEIPGTTRYSVPVFSEGDYSPASLVKKYGVSSDTAVDSISSTNAKPTGFVHAYEAIARSAAGNGSFRKITDHIIQHPDRPILFHCTLGKDRTGVFAALLLSLCGVPNETIVEDYAMTTEGFGAWREHLVQRLLQRKDAATREEAESIIASHPETMKAFLEDVVAAKFGSARNYFVQQCGLTQADVDKLIHTLVFTN

SEQ ID NO: 13 - DNA, 957 bp, synthetic construct
  source 1..957: mol_type = other DNA
atgtcaacctcaaaccccctcccctccccacctttccacaacgtccccaacatcgccaacctccgagacgccgctctcttcccgctctccacaccctctggacccctcaaaccgtctctcctcttccgctccgccgacgtctccaagctgcttcccgagaactggtcctcgctctctagcctaggtgtcacgcacgtgttcgacctgcgctctgtgcccgaagttggcttttcctcatctacgccctccgcctctttgccagcctgggtgtcgtccatgcaggatgcggggatcaacaggacgtgggtccccgtcttcgcggaggcggactattcgcctgagggacttgcgaagcggtatgtgaagtatatggatgaggcggtggagggattcgtgtctgcatatcatgacattcttctctctgcgggcccggcgtatcgcgctatcttgctctatctgatcgattcgcccaacaagggcgtgttggtgcactgcacagccggcaaagatcgtacgggcatcttcttcggcgtcctattcgactacctcggcgttgaccgccaaatcatcgcagatgaatacaacctcactgaactcggcctgggcagcgtgcgggaagaggtcgtggcgcgcttactcaagtccgttgcgttccgcaattatatgaacacgaagcagacaggcaaagaactgagcacggaggaaattggacgtttgattcaggaggagaaggagggcaagacgccagatgtggaggaggagctgagtcccgagacgcgcgaggtggggcgccaggctgcgctgcgcatggtgggggcgcgcaaggagacgatgcttaaggcgctcgagatggtggatagtgagtttggtggcgcggagaagtatctgagggagtattgtgggctgggcgatgaggagctggagaagctgaggaagaatctggtgcagggcgcgtga

SEQ ID NO: 14 - DNA, 957 bp, synthetic construct
  source 1..957: mol_type = other DNA
atgtcaacttcaaaccccttacccagcccaccattccataacgtcccaaacatcgctaacttgagagacgccgccttgttcccattgtccactccttccggtccattgaagccttctttattgttcagatccgctgacgtctctaagttgttgccagaaaactggtcctctttgtcttccttgggtgttactcacgttttcgatttgagatctgttccagaagttggtttctcttcctctactccatctgcctccttgccagcttgggtttcttctatgcaagacgccggtattaacagaacttgggttcctgttttcgctgaagccgattactctccagaaggtttagctaagagatacgttaaatacatggacgaagctgtcgaaggtttcgtttctgcttaccacgatattttgttgtccgctggtccagcttacagagctatcttgttgtacttgattgattctccaaacaagggtgtcttggttcactgcactgctggtaaggacagaaccggtatctttttcggtgtcttgtttgattacttgggtgttgacagacaaatcattgccgacgagtacaacttgactgaattgggtttaggttctgttagagaagaagttgttgccagattgttgaagtccgttgccttcagaaactacatgaacactaaacaaactggtaaggaattgtctactgaggaaattggtagattgattcaagaagaaaaggaaggtaagactccagacgtcgaagaagaattgtctccagaaaccagagaagttggtagacaagctgctttaagaatggttggtgctagaaaagaaactatgttgaaggccttggaaatggtcgactctgagtttggtggtgctgaaaagtacttgagagaatactgcggtttgggtgacgaagaattggaaaagttgagaaagaacttagtccaaggtgcttaa

SEQ ID NO: 15 - protein, 318 aa, synthetic construct
MSTSNPLPSPPFHNVPNIANLRDAALFPLSTPSGPLKPSLLFRSADVSKLLPENWSSLSSLGVTHVFDLRSVPEVGFSSSTPSASLPAWVSSMQDAGINRTWVPVFAEADYSPEGLAKRYVKYMDEAVEGFVSAYHDILLSAGPAYRAILLYLIDSPNKGVLVHCTAGKDRTGIFFGVLFDYLGVDRQIIADEYNLTELGLGSVREEVVARLLKSVAFRNYMNTKQTGKELSTEEIGRLIQEEKEGKTPDVEEELSPETREVGRQAALRMVGARKETMLKALEMVDSEFGGAEKYLREYCGLGDEELEKLRKNLVQGA

SEQ ID NO: 16 - DNA, 879 bp, synthetic construct
  source 1..879: mol_type = other DNA
atggctgaactattcgacaaggcacccgaactgcccccaccattcgtaaatgtaacaggcatcgccaacttccgcgacattggcggctacagcaccaccgacaagcactcaatccgccgaggtctcgtcttccgcgccgccgacccgagcaaagtgcaaccagaaggccttgccaaattgaaagagctaggagtgaagaaggtattcgatcttcgcagtatacccgagatccacaagcaaggtccggaatggcagggtgttgaagttgagaaggaggtgttcatcaccaagtccgaaggtagtgttgaagctgtcccagaaggagatattgaaaggatatggtgtccggtattccgaaacacggattatggtcctgagcaagtcgccatcaggttccagaattatgctaagaaggggagcgagggattcgtaaaagcgtacgcagatatcatggcgaatggccctactgcatacagatccatcctcacccatctcgctcaaccaaacccttcgccatgtataatccactgcacggcaggcaaagaccgcaccggcgtcatctgcgcgatcctgtatctcctctgctccgtccccgccaccacagtagcaaaagaatactccctcaccgacaccggactacagcatttagtccctctattcacagaacgcctgctcaagaacccagcattagttggcaacgaggaaggagtcaagaatatgatttccgcacagccggagaagatggctgcgactattgagatgatgaaggaggtttatggtggggcggaggggtatgtgagggaggttgttggattgagtggggagcaaattgagcagatcaggaagaatcttttgagtgacgaggaggctgtcttttga

SEQ ID NO: 17 - DNA, 879 bp, synthetic construct
  source 1..879: mol_type = other DNA
atggcagaattattcgacaaggcacctgaattgccacctcctttcgtcaatgttactggtattgctaacttccgtgacatcggtggttactctactactgacaagcactctattcgtagaggtttggtcttcagagctgctgacccatctaaagttcaacctgaaggtttggctaagttgaaggaattgggtgtcaagaaggtcttcgatttgagatccatcccagaaatccacaagcaaggtccagaatggcaaggtgtcgaagttgaaaaggaagttttcatcactaagtctgaaggttccgtcgaagccgttccagaaggtgatattgaaagaatctggtgtcctgttttccgtaataccgactatggtccagaacaagttgctattagattccaaaactacgctaagaaaggttccgaaggttttgttaaagcctatgccgacatcatggccaacggtccaactgcttacagatctatcttgacccatttggctcaacctaatccatctccttgtatcattcactgtactgctggtaaggatagaactggtgttatctgcgctattttgtacttgttatgttccgttccagctaccactgttgctaaggaatattctttgactgacactggtttacaacacttggttccattatttaccgaaagattgttgaagaaccctgccttggttggtaacgaagaaggtgttaagaacatgatctccgctcaaccagaaaagatggctgctactattgaaatgatgaaggaagtctatggtggtgctgagggttacgtcagagaagttgtcggtttgtctggtgaacaaatcgaacaaattcgtaagaacttgttgtccgacgaagaagccgttttctaa

SEQ ID NO: 18 - protein, 292 aa, synthetic construct
MAELFDKAPELPPPFVNVTGIANFRDIGGYSTTDKHSIRRGLVFRAADPSKVQPEGLAKLKELGVKKVFDLRSIPEIHKQGPEWQGVEVEKEVFITKSEGSVEAVPEGDIERIWCPVFRNTDYGPEQVAIRFQNYAKKGSEGFVKAYADIMANGPTAYRSILTHLAQPNPSPCIIHCTAGKDRTGVICAILYLLCSVPATTVAKEYSLTDTGLQHLVPLFTERLLKNPALVGNEEGVKNMISAQPEKMAATIEMMKEVYGGAEGYVREVVGLSGEQIEQIRKNLLSDEEAVF

SEQ ID NO: 19 - DNA, 936 bp, synthetic construct
  source 1..936: mol_type = other DNA
atgtctaatgacaccactaccacggcttctgccggaacagcaacttcttcgcggtttctttccgtggggggagttgtgaacttccgtgaactgggcggttacccatgtgattctgtccctcctgctcctgcctcaaacggctcaccggacaatgcatctgaagcgaccctttgggttggccactcgtccattcggcctggatttctgtttcgatcggcacagccgtctcagattaccccggccggtattgagacattgatccgccagcttggcatccagacaatttttgactttcgttcaaggacggaaattgagcttgttgccactcgctatcctgattcgctacttgagatacctggcacgactcgctattccgtgcccgtcttctcggaaggcgactattccccagcgtcattagtcaagaggtacggagtgtcctccgatactgcaaccgattccacttcctccaaaagtgctaagcctacaggattcgtccacgcatatgaggctatcgcacgcagtgcagcagaaaacggcagttttcgtaagataacggaccacataatacaacatccggaccggcctattctgtttcactgtacactggggaaagaccgaaccggtgtgtttgcagcattgttattgagtctttgcggggtaccagacgagacgatagttgaagactatgctatgactaccgagggatttggagcctggcgggaacatctaattcaacgcttgctacaaaggaaggatgcagctacgcgcgaggatgcagaatccattattgccagccccccggagactatgaaggcttttctagaagatgtggtagcagccaagttcgggggtgctcgaaattactttatccagcactgtggatttacggaagctgaggttgataagttaagccatacactggccattacgaattga

SEQ ID NO: 20 - DNA, 936 bp, synthetic construct
  source 1..936: mol_type = other DNA
atgtccaatgatactacgacaactgcttccgccggtactgctacttcctccagattcttgtccgtcggtggtgttgttaatttcagagaattaggtggttacccttgtgattctgttccaccagctccagcctccaatggttcccctgacaacgcttctgaggctactttgtgggttggtcactcttccattagaccaggttttttgtttagatccgctcaaccttctcaaatcactccagccggtattgaaactttgatcagacaattgggtattcaaactatcttcgatttcagatctagaactgaaattgaattggttgctactagatacccagattctttgttagaaattccaggtaccactcgttactccgtcccagtcttctccgaaggtgactattccccagcttctttggttaaaagatacggtgtttcttctgacaccgccactgattccacttcctctaagtccgctaaacctaccggtttcgttcacgcttatgaagccatcgccagatccgccgctgaaaacggttctttccgtaagatcaccgaccatatcattcaacacccagacagaccaattttgtttcattgtactttgggtaaggatagaactggtgtcttcgctgctttattgttgtctttatgtggtgtccctgatgaaactattgttgaagattacgccatgactactgaaggttttggtgcttggagagaacacttaatccaaagattgttgcaaagaaaggatgctgctactagagaagacgctgaatctatcattgcttccccaccagaaactatgaaggctttcttggaagatgttgttgctgctaagtttggtggtgctagaaactacttcatccaacactgcggtttcactgaagctgaagtcgacaagttgtctcatactttggctattactaactaa

SEQ ID NO: 21 - protein, 311 aa, synthetic construct
MSNDTTTTASAGTATSSRFLSVGGVVNFRELGGYPCDSVPPAPASNGSPDNASEATLWVGHSSIRPGFLFRSAQPSQITPAGIETLIRQLGIQTIFDFRSRTEIELVATRYPDSLLEIPGTTRYSVPVFSEGDYSPASLVKRYGVSSDTATDSTSSKSAKPTGFVHAYEAIARSAAENGSFRKITDHIIQHPDRPILFHCTLGKDRTGVFAALLLSLCGVPDETIVEDYAMTTEGFGAWREHLIQRLLQRKDAATREDAESIIASPPETMKAFLEDVVAAKFGGARNYFIQHCGFTEAEVDKLSHTLAITN

SEQ ID NO: 22 - DNA, 2892 bp, synthetic construct
  source 1..2892: mol_type = other DNA
atgagcccaatggatttacaagaatcagcggcagctttggtgcggcagttgggggagagagtcgaagatcgccgtggttttggattcatgagccctgccatctatgataccgcatgggtctctatgattagcaagacaatcgatgaccaaaaaacatggttgtttgcagaatgtttccagtacattctttctcatcagctcgaagacggtggttgggcaatgtatgcatctgaaatcgacgccatcctaaacacttcggcctcattactatcattaaagagacatctttcaaatccctatcaaattacatctatcacacaagaggatctgtccgcccgcattaacagggctcagaatgctttacagaagcttctcaatgagtggaatgtcgacagcacgctccacgtgggattcgagatcctagttccggccctactcaggtatctcgaagatgagggcatcgcttttgctttttctggtagagagcgcctgcttgagattgagaaacagaaattatcaaagttcaaagcacagtatctataccttccaatcaaagtgacagctttgcattctctggaagcgttcataggcgccattgagtttgataaagtcagtcaccacaaagtcagcggtgcgttcatggcatctccatcatccacagcagcttacatgatgcatgcgacacaatgggatgatgaatgcgaggattacctacgccacgtcattgctcatgcatctgggaaaggatccggaggtgttccaagcgcttttccttccaccatctttgaaagcgtttggcctctatcaactctgctaaaggtgggatatgatctcaactcggcaccttttatcgaaaaaatcagatcatacttgcatgatgcatatattgctgaaaagggaattctcggcttcactccttttgttggcgctgatgcagatgataccgctaccaccatattggtgctcaatcttttgaaccaaccagtctcagtcgacgcgatgttgaaggaatttgaagaagaacatcacttcaaaacctactctcaggagcgcaatcctagtttctcggccaattgtaacgttcttcttgccttactatacagtcaagagccatcgctttatagcgcgcagatcgaaaaagctataaggttcctctataagcaattcacagattcagaaatggacgttcgagacaaatggaatctatcaccatactattcttggatgctcatgacacaagccatcacgcggttgacgactcttcagaagacttcgaaactttcaacattgagagatgattctatcagcaaaggcttgattagtctgctgtttaggatagcttctaccgtggttaaagaccaaaagccaggaggttcttggggcactcgagcttcgaaagaagagactgcctacgcagtgttgattctcacatatgctttctacctcgatgaggttacggagtcgttgcggcatgatatcaagatcgccattgagaatggttgctcattcctatctgaaagaaccatgcagtccgattcggagtggctttgggttgagaaagtcacatataaatcagaggttctttcggaagcatatatcttggccgctcttaaacgggcagctgacttacccgacgaaaatgcagaagcagcccccgtcataaatggaatttctacaaatggatttgagcataccgatagaattaacggcaagcttaaagtcaatggtaccaacggtacaaatggcagtcatgagacaaacggtatcaacggtacgcatgaaattgaacagatcaatggcgtcaacggcacgaatggtcactctgatgtgcctcacgatacaaatggctgggtagaagagccgaccgccatcaatgagacaaatggccactacgtgaatggcacgaatcacgagactccccttaccaacggcatttccaatggagattctgtttccgttcatacagaccactcggacagttactatcagcgcagtgattggacagccgacgaagaacaaattcttctcggtccatttgactacctggagagcctgccaggcaagaatatgcgctcacaactgattcaatcattcaacacatggctcaaagtcccaactgagagcttggatgttattattaaggtgatttcaatgttgcatacggcctctctcttgatcgatgatattcaggatcaatcaatactccgccgcgggcaacctgtagcgcacagcatctttggcacagcgcaagcaatgaactcagggaattatgtctactttctagcccttagggaggttcagaaactacaaaacccgaaagccatcagtatttatgttgactctttgattgatcttcaccgtggccaaggcatggagcttttctggcgggattctctcatgtgcccaaccgaagagcagtaccttgacatggtcgcaaacaaaactggcggcctgttttgccttgctatccaattgatgcaagctgaagccactatccaagtcgacttcataccacttgtccgactactcggcatcatcttccagatttgtgatgattacttgaatctgaagtctacggcctatacagacaacaaagggttgtgtgaggatttgacagagggcaaattctcttttcctatcatccatagcattcgatccaaccctggcaaccgacagctaatcaacatcttgaagcaaaagccacgtgaagacgacatcaaacgctatgctctatcctatatggaaagcaccaactcatttgagtatactcggggtgtcgttagaaaactgaagaccgaggcaatcgatactattcaaggcttggagaagcacggcctggaagagaatattggcattcgaaagatactagctcgcatgtcccttgagctatga

SEQ ID NO: 23 - DNA, 2892 bp, synthetic construct
  source 1..2892: mol_type = other DNA
atgtcaccgatggaccttcaggagagtgccgctgctttggtccgtcaattaggtgaaagagtcgaagatcgtagaggtttcggttttatgtccccagccatttatgacactgcctgggtttctatgatttccaagaccattgatgatcaaaagacttggttgttcgccgaatgctttcaatacattttgtctcaccaattagaagacggtggttgggctatgtacgcctccgaaatcgatgctattttgaacacctctgcctctttgttgtccttgaaaagacacttatctaacccataccaaattacttctatcactcaagaagatttgtctgctagaatcaacagagctcaaaacgctttgcaaaagttgttgaacgagtggaacgttgattctaccttgcatgttggtttcgaaatcttagttccagccttgttgagatacttagaagatgaaggtattgctttcgccttctctggtagagaaagattgttggaaatcgaaaagcaaaagttgtctaagttcaaagctcaatacttgtacttaccaattaaggtcaccgctttacattccttggaagctttcattggtgccatcgaatttgacaaggtttctcatcataaggtttccggtgctttcatggcttctccatcctctactgctgcttatatgatgcacgccactcaatgggatgatgaatgtgaggactacttaagacacgtcattgcccatgcttctggtaaaggttctggtggtgtcccttctgctttcccatccaccatctttgaatctgtttggccattatctaccttgttaaaagtcggttatgatttgaactctgctccattcatcgaaaagatcagatcttacttgcacgacgcctacattgctgaaaaaggtatcttaggttttactccatttgttggtgccgatgctgacgacaccgctactactatcttggttttgaacttgttgaaccaacctgtctccgttgatgctatgttgaaagaattcgaagaggagcatcactttaagacctattctcaagaacgtaacccatctttctccgctaactgtaacgttttgttggctttgttgtactcccaagagccatccttatattctgctcaaattgaaaaggccattcgtttcttgtacaaacaattcactgactctgaaatggacgttagagataagtggaacttgtctccatactactcttggatgttgatgacccaagccatcacccgtttaactaccttacaaaagacttccaaattgtccaccttgagagatgactccatttctaagggtttgatctctttgttattccgtatcgcttctactgttgtcaaggaccaaaaaccaggtggttcttggggtactagagcctccaaagaagaaactgcttacgccgttttgatcttgacttacgctttttacttagacgaagttaccgaatctttgcgtcatgacatcaagattgccattgaaaacggttgctctttcttgtctgagagaactatgcaatctgactccgaatggttgtgggtcgagaaggtcacttacaaatccgaagtcttgtccgaagcttacattttggctgccttaaagagagctgccgatttgccagatgaaaatgctgaagctgctccagttattaatggtatctctactaacggtttcgaacacactgatagaattaacggtaagttgaaggttaacggtactaacggtaccaacggttcccatgaaactaacggtattaacggtacccacgaaattgaacaaatcaacggtgtcaacggtactaatggtcattctgatgttccacacgatactaacggttgggtcgaggaaccaactgctatcaacgaaactaacggtcactatgttaacggtaccaatcacgaaactccattaaccaacggtatttctaatggtgactctgtttccgttcatactgaccactctgactcttattatcaacgttctgattggactgctgacgaagaacaaatcttgttaggtccatttgactacttggaatctttgccaggtaaaaacatgcgttctcaattgatccaatccttcaatacctggttgaaggtcccaactgaatctttggacgtcatcatcaaggttatttctatgttgcataccgcctccttattgattgatgatattcaagaccaatccatcttgcgtcgtggtcaacctgtcgctcactctatcttcggtactgctcaagccatgaattctggtaactacgtctacttcttagctttaagagaagttcaaaagttgcaaaacccaaaggctatttctatttacgtcgactctttgattgacttgcacagaggtcaaggtatggaattgttctggagagattctttaatgtgtcctactgaagaacaatacttggatatggttgctaacaagaccggtggtttgttctgcttggccattcaattgatgcaagctgaagccactattcaagtcgacttcattccattggtcagattgttgggtatcatcttccaaatttgtgacgattacttgaacttgaagtctactgcctacaccgataacaagggtttgtgtgaagatttgactgaaggtaagttttctttcccaatcatccactctattagatctaacccaggtaaccgtcaattgatcaacatcttgaagcaaaaaccaagagaagacgacattaagagatacgccttgtcttacatggaatccaccaactctttcgaatacactagaggtgttgttagaaaattgaagaccgaagctatcgataccatccaaggtttagaaaagcacggtttggaggagaatattggtatccgtaagattttggcccgtatgtccttggaattgtaa

SEQ ID NO: 24 - protein, 963 aa, synthetic construct
MSPMDLQESAAALVRQLGERVEDRRGFGFMSPAIYDTAWVSMISKTIDDQKTWLFAECFQYILSHQLEDGGWAMYASEIDAILNTSASLLSLKRHLSNPYQITSITQEDLSARINRAQNALQKLLNEWNVDSTLHVGFEILVPALLRYLEDEGIAFAFSGRERLLEIEKQKLSKFKAQYLYLPIKVTALHSLEAFIGAIEFDKVSHHKVSGAFMASPSSTAAYMMHATQWDDECEDYLRHVIAHASGKGSGGVPSAFPSTIFESVWPLSTLLKVGYDLNSAPFIEKIRSYLHDAYIAEKGILGFTPFVGADADDTATTILVLNLLNQPVSVDAMLKEFEEEHHFKTYSQERNPSFSANCNVLLALLYSQEPSLYSAQIEKAIRFLYKQFTDSEMDVRDKWNLSPYYSWMLMTQAITRLTTLQKTSKLSTLRDDSISKGLISLLFRIASTVVKDQKPGGSWGTRASKEETAYAVLILTYAFYLDEVTESLRHDIKIAIENGCSFLSERTMQSDSEWLWVEKVTYKSEVLSEAYILAALKRAADLPDENAEAAPVINGISTNGFEHTDRINGKLKVNGTNGTNGSHETNGINGTHEIEQINGVNGTNGHSDVPHDTNGWVEEPTAINETNGHYVNGTNHETPLTNGISNGDSVSVHTDHSDSYYQRSDWTADEEQILLGPFDYLESLPGKNMRSQLIQSFNTWLKVPTESLDVIIKVISMLHTASLLIDDIQDQSILRRGQPVAHSIFGTAQAMNSGNYVYFLALREVQKLQNPKAISIYVDSLIDLHRGQGMELFWRDSLMCPTEEQYLDMVANKTGGLFCLAIQLMQAEATIQVDFIPLVRLLGIIFQICDDYLNLKSTAYTDNKGLCEDLTEGKFSFPIIHSIRSNPGNRQLINILKQKPREDDIKRYALSYMESTNSFEYTRGVVRKLKTEAIDTIQGLEKHGLEENIGIRKILARMSLEL

SEQ ID NO: 25 - DNA, 500 bp, synthetic construct
  source 1..500: mol_type = other DNA
tggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactata

SEQ ID NO: 26 - DNA, 501 bp, synthetic construct
  source 1..501: mol_type = other DNA
catcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataa

SEQ ID NO: 27 - DNA, 509 bp, synthetic construct
  source 1..509: mol_type = other DNA
ggcttaagtaggttgcaatttctttttctattagtagctaaaaatgggtcacgtgatctatattcgaaaggggcggttgcctcaggaaggcaccggcggtctttcgtccgtgcggagatatctgcgccgttcaggggtccatgtgccttggacgatattaaggcagaaggcagtatcggggcggatcactccgaaccgagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacataataaaaaaaataattctttcata

SEQ ID NO: 28 - DNA, 498 bp, synthetic construct
  source 1..498: mol_type = other DNA
ttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattcttgaaagaatgaaatcgccatgccaagccatcacacggtcttttatgcaattgattgaccgcctgcaacacataggcagtaaaatttttactgaaacgtatataatcatcataagcgacaagtgaggcaacacctttgttaccacattgacaaccccaggtattcatacttcctattagcggaatcaggagtgcaaaaagagaaaataaaagtaaaaaggtagggcaacacatagt

SEQ ID NO: 29 - DNA, 501 bp, synthetic construct
  source 1..501: mol_type = other DNA
ggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 30 - DNA, 489 bp, synthetic construct
  source 1..489: mol_type = other DNA
gcgacacagagatgacagacggtggcgcaggatccggtttaaacgaggatcccttaagtttaaacaacaacagcaagcaggtgtgcaagacactagagactcctaacatgatgtatgccaataaaacacaagagataaacaacattgcatggaggccccagaggggcgattggtttgggtgcgtgagcggcaagaagtttcaaaacgtccgcgtcctttgagacagcattcgcccagtattttttttattctacaaaccttctataatttcaaagtatttacataattctgtatcagtttaatcaccataatatcgttttctttgtttagtgcaattaatttttcctattgttacttcgggcctttttctgttttatgagctattttttccgtcatccttccccagattttcagcttcatctccagattgtgtctacgtaatgcacgccatcattttaagagaggacagagaagcaagcctcctgaaag

SEQ ID NO: 31 - DNA, 1253 bp, synthetic construct
  source 1..1253: mol_type = other DNA
gatgatggacactagtgtgtcgagaatgtatcaactatatatagtcctaatgccacacaaatatgaagtgggggaagcccattcttaatccggctcaattttggtgcgtgatcgcggcctatgtttgcttccagaaaaagcttagaataatatttctcacctttgatggaatgctcgcgagtgctcgttttgattaccccatatgcattgttgcagcatgcaagcactattgcaagccacgcatggaagaaatttgcaaacacctatagccccgcgttgttgaggaggtggacttggtgtaggaccataaagctgtgcactactatggtgagctctgtcgtctggtgaccttctatctcaggcacatcctcgtttttgtgcatgaggttcgagtcacgcccacggcctattaatccgcgaaataaatgcgaaatctaaattatgacgcaaggctgagagattctgacacgccgcatttgcggggcagtaattatcgggcagttttccggggttcgggatggggtttggagagaaagttcaacacagaccaaaacagcttgggaccacttggatggaggtccccgcagaagagctctggcgcgttggacaaacattgacaatccacggcaaaattgtctacagttccgtgtatgcggatagggatatcttcgggagtatcgcaataggatacaggcactgtgcagattacgcgacatgatagctttgtatgttctacagactctgccgtagcagtctagatataatatcggagttttgtagcgtcgtaaggaaaacttgggttacacaggtttcttgagagccctttgacgttgattgctctggcttccatccaggccctcatgtggttcaggtgcctccgcagtggctggcaagcgtgggggtcaattacgtcacttctattcatgtaccccagactcaattgttgacagcaatttcagcgagaattaaattccacaatcaattctcgctgaaataattaggccgtgatttaattctcgctgaaacagaatcctgtctggggtacagataacaatcaagtaactattatggacgtgcataggaggtggagtccatgacgcaaagggaaatattcattttatcctcgcgaagttgggatgtgtcaaagcgtcgcgctcgctatagtgatgagaatgtctttagtaagcttaagccatataaagaccttccgcctccatatttttttttatccctcttgacaatattaattcctt

SEQ ID NO: 32 - DNA, 1253 bp, synthetic construct
  source 1..1253: mol_type = other DNA
aaggaattaatattgtcaagagggataaaaaaaaatatggaggcggaaggtctttatatggcttaagcttactaaagacattctcatcactatagcgagcgcgacgctttgacacatcccaacttcgcgaggataaaatgaatatttccctttgcgtcatggactccacctcctatgcacgtccataatagttacttgattgttatctgtaccccagacaggattctgtttcagcgagaattaaatcacggcctaattatttcagcgagaattgattgtggaatttaattctcgctgaaattgctgtcaacaattgagtctggggtacatgaatagaagtgacgtaattgacccccacgcttgccagccactgcggaggcacctgaaccacatgagggcctggatggaagccagagcaatcaacgtcaaagggctctcaagaaacctgtgtaacccaagttttccttacgacgctacaaaactccgatattatatctagactgctacggcagagtctgtagaacatacaaagctatcatgtcgcgtaatctgcacagtgcctgtatcctattgcgatactcccgaagatatccctatccgcatacacggaactgtagacaattttgccgtggattgtcaatgtttgtccaacgcgccagagctcttctgcggggacctccatccaagtggtcccaagctgttttggtctgtgttgaactttctctccaaaccccatcccgaaccccggaaaactgcccgataattactgccccgcaaatgcggcgtgtcagaatctctcagccttgcgtcataatttagatttcgcatttatttcgcggattaataggccgtgggcgtgactcgaacctcatgcacaaaaacgaggatgtgcctgagatagaaggtcaccagacgacagagctcaccatagtagtgcacagctttatggtcctacaccaagtccacctcctcaacaacgcggggctataggtgtttgcaaatttcttccatgcgtggcttgcaatagtgcttgcatgctgcaacaatgcatatggggtaatcaaaacgagcactcgcgagcattccatcaaaggtgagaaatattattctaagctttttctggaagcaaacataggccgcgatcacgcaccaaaattgagccggattaagaatgggcttcccccacttcatatttgtgtggcattaggactatatatagttgatacattctcgacacactagtgtccatcatc

SEQ ID NO: 33 - DNA, 665 bp, synthetic construct
  source 1..665: mol_type = other DNA
gcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataat

SEQ ID NO: 34 - DNA, 785 bp, synthetic construct
  source 1..785: mol_type = other DNA
attatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataa

SEQ ID NO: 35 - DNA, 875 bp, synthetic construct
  source 1..875: mol_type = other DNA
ttatgtattttagttacgcttgactgatgtacatttgagattatcaaaaaaactgcttaagagatagatggtttaattttttagagacgtattaatggaactttttataccttgcccagagcgcctcaagaaaatgatgctgaaagaagaattgaggaaggaactactcatcttacgttgtttgtatcatcccacgatccaaatcatgttacctacgttaggtacgctaggaactgaaaaaagaaaagaaaagtatgcgttatcactcttcgagccaattcttaattgtgtggggtccgcgaaaacttccggataaatcctgtaaacttaaacttaaaccccgtgtttagcgaaattttcaacgaagcgcgcaataaggagaaatattatataaaagcgagagtttaagcgaggttgcaagaatctctacggtacagatgcaacttactatagccaaggtctattcgtattggtatccaagcagtgaagctactcaggggaaaacatattttcagagatcaaagttatgtcagtctctttttcatgtgtaacttaacgtttgtgcaggtatcataccggcctccacataatttttgtggggaagacgttgttgtagcagtctccttatactctccaacaggtgtttaaagacttcttcaggcctcatagtctacatctggagacaacattagatagaagtttccacagaggcagctttcaatatactttcggctgtgtacatttcatcctgagtgagcgcatattgcataagtactcagtatataaagagacacaatatactccatacttgttgtgagtggttttagcgtattcagtataacaataagaattacatccaagactattaattaact

SEQ ID NO: 36 - DNA, 875 bp, synthetic construct
  source 1..875: mol_type = other DNA
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa

SEQ ID NO: 37 - DNA, 963 bp, synthetic construct
  source 1..963: mol_type = other DNA
atgttgacctctagcaaatcaattgaatccttccccaagaatgttcaaccttatggcaagcattatcaaaatggcttggaacctgttggaaaaagccaagaagatattctcttggagccattccactatctctgttcgaatcctggtaaagatgtccgaaccaagatgattgaagcgttcaatgcttggctgaaagtacccaaggacgatttgatcgtcatcacacgtgtgattgaaatgcttcatagtgctagtttgttaattgatgatgtggaagatgattccgtgttgcgtcgtggtgttcctgcagctcatcatatatatggtactcctcaaactatcaattgtgctaattacgtgtactttcttgcactgaaagaaattgccaagttgaacaagcccaacatgattactatctataccgatgaattgatcaatttgcacagagggcaaggaatggaattgttttggcgtgacaccttaacttgtcctacagagaaagaatttcttgacatggtaaacgacaaaactggtggcctcttgagattagctgtgaaacttatgcaagaagctagtcaatcgggaactgattatacgggactcgtaagtaagattggtatccatttccaagtacgcgacgattatatgaatttgcagtcaaaaaactatgctgacaacaaaggattctgcgaagacttgacagaaggaaaattctctttccctattatacattcaatccgctctgacccaagcaatcgccagcttttgaacattttaaaacagcgcagtagctctatcgaactcaagcaatttgccttgcagctactggaaaacacaaacactttccaatactgtcgtgatttcttacgtgtcttggaaaaggaagctagagaagaaattaagcttttagggggtaacatcatgttggagaaaattatggatgtcttgagtgtcaatgaataa

SEQ ID NO: 38 - DNA, 963 bp, synthetic construct
  source 1..963: mol_type = other DNA
atgttgacatcttctaagtccatcgaatctttcccaaagaacgttcaaccatacggtaaacactatcaaaacggtttagaaccagtcggtaagtctcaagaagacatcttgttggaacctttccactacttatgttctaatccaggtaaggatgttagaaccaagatgattgaagctttcaacgcctggttgaaagtcccaaaggacgatttgattgttatcaccagagtcattgaaatgttgcactccgcttctttgttgattgatgacgtcgaggacgattctgtcttgagaagaggtgtcccagccgcccaccatatctacggtacccctcaaaccatcaactgcgctaactacgtttatttcttggccttgaaagaaatcgccaagttgaacaagccaaatatgattactatttataccgatgaattgatcaacttgcacagaggtcaaggtatggaattgttctggcgtgataccttgacctgcccaactgagaaagagtttttggatatggttaacgataagactggtggtttgttgagattggccgtcaagttgatgcaagaggcttctcaatctggtaccgactatactggtttggtttctaagatcggtatccattttcaagttagagatgactacatgaacttgcaatccaaaaactacgccgataataagggtttctgtgaagatttgaccgaaggtaagttctcctttccaattattcactctatcagatctgacccatccaacagacaattattgaatattttgaagcaaagatcttcttctattgaattgaaacaattcgctttacaattgttagaaaacactaacacttttcaatactgtagagatttcttgagagttttggaaaaggaagccagagaagagatcaaattattgggtggtaacatcatgttggaaaagattatggacgtcttgtctgttaatgaataa

SEQ ID NO: 39 - protein, 320 aa, synthetic construct
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 40 - DNA, 2258 bp, synthetic construct
  source 1..2258: mol_type = other DNA
atgtcatggatgaacaacggtaaaaaccttaactgccaacttactcacaagaaaatatcgaaagtagccgagattcgagttgccacggtgaacgcgccgccggtgcacgatcaagacgattccacagaaaatcagtgccatgacgcggtgaataatattgaggatccgatcgaatacataagaacgctgctgaggacgacaggggacggccgaataagtgtgtcgccgtatgacactgcgtgggtcgctctgatcaaggacttgcaaggacgcgatgcccccgagtttccgtcgagcctggagtggatcatacagaatcagctggccgatgggtcgtggggcgatgccaagttcttctgtgtgtatgatcgcctcgtgaatacgatagcatgcgtggtggccttgagatcatgggatgttcatgctgaaaaggtggaaagaggagtgagatacatcaatgaaaatgtggaaaagcttagagatggaaatgaggaacacatgacttgtgggttcgaagtggtgtttcctgcgcttctgcagagagctaagagcttagggatccaagatcttccctatgatgctcccgtcattcaagagatatatcactccagggaacaaaagttgaaaaggattccactggagatgatgcacaaagtgccaacttctttattatttagtctggaagggctggagaatttggagtgggataagcttttgaaactgcagtcagctgatggctctttcctcacttctccctcctccactgccttcgcttttatgcaaactcgtgatcctaaatgctaccaattcatcaaaaacactattcaaactttcaacggaggagcaccacacacttatcctgtcgatgtttttggaagactttgggcaatcgacaggctgcagcgcctcgggatttctcgcttctttgagtccgagattgctgattgcatcgcccacatccacaggttttggacagagaagggagttttcagtggaagagaatcagagttttgcgacattgatgatacatccatgggagtccgactcatgagaatgcatggatacgatgttgatccaaatgtattgaagaacttcaaaaaggatgacaagttttcatgctacggtggacagatgattgagtctccgtctcccatttacaatctctacagggcttcccaactccgcttccccggtgagcaaattctcgaagatgccaacaaatttgcctacgatttcttacaagaaaagcttgcccacaaccagattcttgataaatgggttatatctaagcacttgcctgatgagataaaactgggactggagatgccgtggtacgccaccctaccccgcgtggaggcaagatactacatacagtactatgctggttcaggcgatgtatggatcggaaagactctctacaggatgcccgagatcagcaacgatacatatcatgagcttgcaaaaacagacttcaagagatgccaagctcagcatcagtttgagtggatttacatgcaagaatggtacgagagttgcaacatggaagaattcgggataagcagaaaggagcttctggttgcttacttcttggcgactgcaagcatattcgagctggagagggctaatgagagaatcgcctgggccaaatcccaaatcatttccaccatcattgcatctttcttcaataaccaaaacacttcaccggaggataaacttgcatttttaacagatttcaaaaatggcaactccacaaacatggctctggtgaccctcactcaattcctagagggattcgacagatacactagccatcagttgaagaatgcctggagcgtatggctgagaaagctgcagcaaggagaaggcaacggcggcgcagacgcagagctcctagtaaacacattgaacatttgtgccggccacattgcctttagggaagaaatactcgcacacaacgactacaagactctctccaacctgactagcaaaatctgtcgacaactttctcaaattcaaaatgaaaaggagttggagacagagggacagaaaacaagcataaaaaacaaggaactggaagaagatatgcaaagactggtgaagttggtgttggagaaatcaagggttggaatcaacagagatatgaagaaaacatttcttgcagtggtaaaaacttattactacaaagcatatcattctgctcaggccatcgacaaccatatgttcaaagtacttttcgaaccagtcgccctcgagtgctg

SEQ ID NO: 41 - DNA, 2382 bp, synthetic construct
  source 1..2382: mol_type = other DNA
atggcctccttatcctctacaatcctcagccgctctccggcggcccgccgcagaattacgccggcgtcggctaagcttcaccggccggaatgtttcgccaccagtgcatggatgggcagcagcagtaaaaacctttctctcagctaccaacttaatcacaagaaaatatcagttgccacagtagatgcgccgcaggtgcatgaccacgacggcactaccgttcatcaaggccatgatgcggtgaagaatattgaggatcccattgaatacatcaggacgttgttgaggacgacgggggacgggagaataagcgtgtcgccgtacgacacggcgtgggtggcgatgatcaaggacgtggaggggcgggacggcccccagttcccctccagcctcgagtggatcgtgcagaatcaactcgaggatggatcgtggggcgatcagaagcttttctgcgtctacgatcgcctcgtcaataccatcgcgtgcgtggtagccttgagatcgtggaatgttcatgctcacaaggtcaaaagaggagtgacgtacatcaaggaaaatgtggataaacttatggagggaaatgaggagcacatgacttgtgggttcgaagtggtgtttccggcgcttctacaaaaagcgaaaagcttaggcatcgaagatcttccttacgattctccggcggtgcaggaggtttatcatgtcagggaacaaaagttgaaaaggattccactggagattatgcacaaaataccgacatcattattatttagtttggaagggctcgaaaatttggattgggacaaacttttgaaactgcagtcagccgacggttccttcctcacctctccctcctccaccgccttcgcgttcatgcaaaccaaggatgaaaaatgctaccaattcatcaagaacacgatagacactttcaacggaggagcgccacacacttatcccgtcgacgtgtttggaaggctctgggcgatcgaccggctgcagcgcctcggaatttcccgcttttttgagccggagattgctgattgcttaagccacatccacaaattttggacggataagggagttttcagtgggagagaatcggagttttgcgacattgacgatacatccatgggaatgaggcttatgaggatgcatggatatgatgttgatccaaatgtgctgaggaatttcaagcagaaagatggtaaattctcttgctacggcgggcagatgatcgagtcgccttctccgatatacaatctttacagagcttctcagctccgatttcccggcgaggaaatcctcgaagatgcgaagagattcgcctacgatttcttgaaagaaaaactagccaacaatcagattctggataaatgggttatttctaagcacttgcctgatgagatcaagctcgggctagagatgccgtggctcgccaccctaccccgcgtcgaggcgaagtactacatccagtactacgccggctccggcgacgtgtggatcggaaagacgctgtacaggatgccggagatcagcaacgacacgtaccacgacctagccaagacggatttcaagagatgccaagcgaagcatcagttcgagtggctctacatgcaagaatggtacgagagctgcggcatcgaggaattcgggataagcagaaaggaccttctgctttcctatttcttggcgaccgcgagcatcttcgagctcgagaggaccaacgagcgaatcgcgtgggccaaatcgcagatcatcgctaagatgatcacttctttcttcaacaaggaaactacgtcggaggaggacaagcgagctcttttgaacgagctcggaaacattaatggcctcaacgacacaaacggcgcagggagagaaggtggggccggtagcattgcgctagcgaccctcactcagttcctcgagggattcgacagatacaccagacaccagctgaaaaatgcttggagcgtatggctgacgcagctgcaacatggcgaagcagacgacgcggagctcctaaccaacacgttgaacatctgcgccggccacatcgccttcagggaagaaatactggcgcacaacgagtacaaagctctctccaacctaaccagcaaaatctgtcgacagctttctttcattcaaagcgaaaaggagatgggagtagagggcgagatcgcagcgaaatcgagcataaaaaacaaggaactcgaagaagacatgcaaatgttggtgaagttggtgcttgagaaatatgggggcatagatagaaatataaagaaagcgtttttagcagttgcgaagacttattattacagagcgtatcatgccgccgacaccatagacacacacatgtttaaagtgcttttcgagccagtcgcgtga

SEQ ID NO: 42 - protein, 786 aa, synthetic construct
MGSLSTMNLNHSPMSYSGILPSSSAKAKLLLPGCFSISAWMNNGKNLNCQLTHKKISKVAEIRVATVNAPPVHDQDDSTENQCHDAVNNIEDPIEYIRTLLRTTGDGRISVSPYDTAWVALIKDLQGRDAPEFPSSLEWIIQNQLADGSWGDAKFFCVYDRLVNTIACVVALRSWDVHAEKVERGVRYINENVEKLRDGNEEHMTCGFEVVFPALLQRAKSLGIQDLPYDAPVIQEIYHSREQKSKRIPLEMMHKVPTSLLFSLEGLENLEWDKLLKLQSADGSFLTSPSSTAFAFMQTRDPKCYQFIKNTIQTFNGGAPHTYPVDVFGRLWAIDRLQRLGISRFFESEIADCIAHIHRFWTEKGVFSGRESEFCDIDDTSMGVRLMRMHGYDVDPNVLKNFKKDDKFSCYGGQMIESPSPIYNLYRASQLRFPGEQILEDANKFAYDFLQEKLAHNQILDKWVISKHLPDEIKLGLEMPWYATLPRVEARYYIQYYAGSGDVWIGKTLYRMPEISNDTYHELAKTDFKRCQAQHQFEWIYMQEWYESCNMEEFGISRKELLVAYFLATASIFELERANERIAWAKSQIISTIIASFFNNQNTSPEDKLAFLTDFKNGNSTNMALVTLTQFLEGFDRYTSHQLKNAWSVWLRKLQQGEGNGGADAELLVNTLNICAGHIAFREEILAHNDYKTLSNLTSKICRQLSQIQNEKELETEGQKTSIKNKELEEDMQRLVKLVLEKSRVGINRDMKKTFLAVVKTYYYKAYHSAQAIDNHMFKVLFEPVA

SEQ ID NO: 43 - protein, 736 aa, synthetic construct
MATVDAPQVHDHDGTTVHQGHDAVKNIEDPIEYIRTLLRTTGDGRISVSPYDTAWVAMIKDVEGRDGPQFPSSLEWIVQNQLEDGSWGDQKLFCVYDRLVNTIACVVALRSWNVHAHKVKRGVTYIKENVDKLMEGNEEHMTCGFEVVFPALLQKAKSLGIEDLPYDSPAVQEVYHVREQKLKRIPLEIMHKIPTSLLFSLEGLENLDWDKLLKLQSADGSFLTSPSSTAFAFMQTKDEKCYQFIKNTIDTFNGGAPHTYPVDVFGRLWAIDRLQRLGISRFFEPEIADCLSHIHKFWTDKGVFSGRESEFCDIDDTSMGMRLMRMHGYDVDPNVLRNFKQKDGKFSCYGGQMIESPSPIYNLYRASQLRFPGEEILEDAKRFAYDFLKEKLANNQILDKWVISKHLPDEIKLGLEMPWLATLPRVEAKYYIQYYAGSGDVWIGKTLYRMPEISNDTYHDLAKTDFKRCQAKHQFEWLYMQEWYESCGIEEFGISRKDLLLSYFLATASIFELERTNERIAWAKSQIIAKMITSFFNKETTSEEDKRALLNELGNINGLNDTNGAGREGGAGSIALATLTQFLEGFDRYTRHQLKNAWSVWLTQLQHGEADDAELLTNTLNICAGHIAFREEILAHNEYKALSNLTSKICRQLSFIQSEKEMGVEGEIAAKSSIKNKELEEDMQMLVKLVLEKYGGIDRNIKKAFLAVAKTYYYRAYHAADTIDTHMFKVLFEPVA

SEQ ID NO: 44 - DNA, 1059 bp, synthetic construct
  source 1..1059: mol_type = other DNA
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 45 - protein, 352 aa, synthetic construct
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

(19) United States
(12) Patent Application Publication
(10) Pub. No.: US 2024/0327875 A1
(43) Pub. Date: Oct. 3, 2024

(54) NOVEL ENZYMES FOR THE PRODUCTION OF E-COPALOL

(71) Applicant: AMYRIS, INC., Emeryville, CA (US)

(72) Inventors: Quinn Mitrovich, Emeryville, CA (US); William E. Draper, Emeryville, CA (US); Andrew Klein, Emeryville, CA (US); Michelle Medina, Emeryville, CA (US)

(21) Appl. No.: 18/578,609

(22) PCT Filed: Jul. 15, 2022
(86) PCT No.: PCT/US2022/073759

§ 371 Date: Jan. 11, 2024

Related U.S. Application Data
(60) Provisional application No. 63/222,597, filed on Jul. 16, 2021.

(51) Int. Cl. C12P 7/04; C12N 1/16; C12N 9/16; C12N 15/81; C12R 1/865

(52) U.S. Cl. CPC C12P 7/04; C12N 1/165; C12N 9/16; C12N 15/81; C12N 2510/00; C12R 2001/865; C12Y 306/01

(57) ABSTRACT

The present disclosure features compositions and methods for producing one or more isoprenoid compounds, such as E-copalol, in a host cell, such as a yeast cell, that is genetically modified to express the enzymes of an isoprenoid biosynthetic pathway, such as a pathway for making E-copalol. Using the compositions and methods of the present invention, the host cell may be genetically modified to express one or more enzymes of an isoprenoid biosynthetic pathway, such as a copalyl-diphosphate (CPP) pyrophosphatase. The host cell may then be cultured in a medium, for example, in the presence of an agent that regulates expression of the one or more enzymes. The host cell may further be incubated for a time sufficient to allow for production of an isoprenoid compound, such as E-copalol, by the host cell. The isoprenoid compound may then be separated from the host cell or from the medium.

### BACKGROUND OF THE INVENTION

Terpenes are a large class of hydrocarbons that are produced in many organisms. They are derived by linking units of isoprene (C₅H₈), and are classified by the number of isoprene units present. Hemiterpenes consist of a single isoprene unit. Isoprene itself is considered the only hemiterpene. Monoterpenes are made of two isoprene units, and have the molecular formula C₁₀H₁₆. Examples of monoterpenes are geraniol, limonene, and terpineol. Sesquiterpenes are composed of three isoprene units, and have the molecular formula C₁₅H₂₄. Examples of sesquiterpenes are farnesene, farnesol and patchoulol. Diterpenes are made of four isoprene units, and have the molecular formula C₂₀NH₃₂. Examples of diterpenes are copalol, cafestol, kahweol, cembrene, and taxadiene. Sesterterpenes are made of five isoprene units, and have the molecular formula C₂₅H₄₀. An example of a sesterterpene is geranylfarnesol. Triterpenes consist of six isoprene units, and have the molecular formula C₃₀H₄₈. Tetraterpenes contain eight isoprene units, and have the molecular formula C₄₀H₆₄. Biologically important tetraterpenes include the acyclic lycopene, the monocyclic gamma-carotene, and the bicyclic alpha- and beta-carotenes. Polyterpenes consist of long chains of many isoprene units. Natural rubber consists of polyisoprene in which the double bonds are in the cis conformation.

When terpenes are chemically modified (e.g., via oxidation or rearrangement of the carbon skeleton) the resulting compounds are generally referred to as terpenoids, which are also known as isoprenoids. Isoprenoids play many important biological roles, for example, as quinones in electron transport chains, as components of membranes, in subcellular targeting and regulation via protein prenylation, as photosynthetic pigments including carotenoids and chlorophyll, as hormones and cofactors, and as plant defense compounds. They are industrially useful as antibiotics, hormones, anticancer drugs, insecticides, and chemicals.

Terpenes are biosynthesized through condensations of isopentenyl pyrophosphate (isopentenyl diphosphate or IPP) and its isomer dimethylallyl pyrophosphate (dimethylallyl diphosphate or DMAPP). Two pathways are known to generate IPP and DMAPP, namely the mevalonate-dependent (MEV) pathway of eukaryotes, and the mevalonate-independent or deoxyxylulose-5-phosphate (DXP) pathway of prokaryotes. Plants use both the MEV pathway and the DXP pathway. IPP and DMAPP in turn are condensed to polyprenyl diphosphates (e.g., geranyl disphosphate or GPP, farnesyl diphosphate or FPP, and geranylgeranyl diphosphate or GGPP) through the action of prenyl disphosphate synthases (e.g., GPP synthase, FPP synthase, and GGPP synthase, respectively).

Traditionally, isoprenoids have been manufactured by extraction from natural sources such as plants, microbes, and animals. However, the yield by way of extraction is usually very low due to a number of profound limitations. First, most isoprenoids accumulate in nature in only small amounts. Second, the source organisms in general are not amenable to the large-scale cultivation that is necessary to produce commercially viable quantities of a desired isoprenoid. Third, the requirement of certain toxic solvents for isoprenoid extraction necessitates special handling and disposal procedures, thus complicating the commercial production of isoprenoids.

The elucidation of the MEV and DXP metabolic pathways has made biosynthetic production of isoprenoids feasible. For instance, microbes have been engineered to overexpress a part of or the entire mevalonate pathway for production of an isoprenoid named amorpha-4,11-diene. Other efforts have focused on balancing the pool of glyceraldehyde-3-phosphate and pyruvate, or on increasing the expression of 1-deoxy-D-xylulose-5-phosphate synthase (dxs) and IPP isomerase (idi).

Nevertheless, given the very large quantities of isoprenoid products needed for many commercial applications, there remains a need for expression systems and fermentation procedures that produce even more isoprenoids than available with current technologies. Optimal redirection of microbial metabolism toward isoprenoid production requires that the introduced biosynthetic pathway is properly engineered both to funnel carbon to isoprenoid production efficiently and to prevent buildup of toxic levels of metabolic intermediates over a sustained period of time. Provided herein are compositions and methods that address this need and provide related advantages as well.

### SUMMARY OF THE INVENTION

Provided herein are compositions and methods for producing one or more isoprenoid compounds, such as E-copalol, in a host cell, such as a yeast cell, that is genetically modified to express the enzymes of an isoprenoid biosynthetic pathway, such as a pathway for making E-copalol. Using the compositions and methods of the present invention, the host cell may be genetically modified to express one or more enzymes of an isoprenoid biosynthetic pathway, such as a copalyl-diphosphate (CPP) pyrophosphatase. The host cell may then be cultured in a medium, for example, in the presence of an agent that regulates expression of the one or more enzymes. The host cell may further be incubated for a time sufficient to allow for production of an isoprenoid compound by the host cell. The isoprenoid compound may then be separated from the host cell or from the medium.

In one aspect, the invention provides for a genetically modified host cell capable of producing E-copalol, wherein the genetically modified host cell contains one or more heterologous nucleic acids that each, independently, encodes an enzyme having an amino acid sequence that is at least 90% identical to the amino acid sequence of SEQ ID NOS. 3, 6, 9, 12, 15, or 18. In an embodiment, the enzyme has the amino acid sequence of SEQ ID NOS. 3, 6, 9, 12, 15, or 18.

In another aspect, the invention provides for a genetically modified host cell capable of producing E-copalol, wherein the genetically modified host cell contains one or more heterologous nucleic acids that each, independently, encodes an enzyme capable of converting copalyl-diphosphate (CPP) to E-copalol. In an embodiment, the enzyme capable of converting CPP to E-copalol is a pyrophosphatase.

In another embodiment, the genetically modified host cell further contains one or more heterologous nucleic acids that each, independently, encodes one or more enzymes of a pathway for making E-copalol. In yet another embodiment, genetically modified host cell further contains one or more heterologous nucleic acids that each, independently, encodes an enzyme having the amino acid sequence of SEQ ID NO. 24, SEQ ID NO. 39, SEQ ID NO. 42, SEQ ID NO. 43, or SEQ ID NO. 45. In yet another embodiment, the genetically modified host cell further contains one or more heterologous nucleic acids that each, independently, encodes an enzyme capable of converting one or more IPP, DMAPP, GPP, FPP, or GGPP into GPP, FPP, GGPP, or CPP. In yet another embodiment, the genetically modified host cell further contains a CPP synthase, an Erg20, a GPP synthase, or a GGPP synthase.

In an embodiment, expression of one or more of the enzymes disclosed herein is under the control of a single transcriptional regulator. In another embodiment, expression of one or more of the enzymes disclosed herein is under the control of multiple transcriptional regulators.

In an embodiment, the genetically modified host cell is a yeast cell or a yeast strain. In yet another embodiment, the yeast cell or the yeast strain is Saccharomyces cerevisiae.

In another aspect, the invention provides for a fermentation composition containing a genetically modified host cell disclosed herein, optionally an overlay, and E-copalol produced by the genetically modified host cell.

In another aspect, the invention provides for a method for producing E-copalol, involving culturing a genetically modified host cell disclosed herein in a medium with a carbon source under conditions suitable for making E-copalol, optionally providing an overlay, and recovering E-copalol from the genetically modified host cell, the overlay, or the medium.

In yet another aspect, the invention provides for a non-naturally occurring enzyme capable of converting CPP to E-copalol and having an amino acid sequence that is at least 90% identical to the amino acid sequence of SEQ ID NOS. 3, 6, 9, 12, 15, or 18. In an embodiment, the non-naturally occurring enzyme has the amino acid sequence of SEQ ID NOS. 3, 6, 9, 12, 15, or 18.

### BRIEF DESCRIPTION OF THE DRAWINGS

FIG. 1 is a schematic showing various enzymatic pathways from the native S. cerevisiae metabolites isopentenyl pyrophosphate (IPP), dimethylallyl pyrophosphate (DMAPP), farnesyl pyrophosphate (FPP), and geranylgeranyl pyrophosphate (GGPP) to E-copalol through the non-native intermediate copalyl-pyrophosphate (CPP).

FIG. 2 is a graph providing relative titers of E-copalol from a 96-well plate experiment in which strains expressing different pyrophosphatase enzymes for the conversion of CPP to E-copalol were cultured on 4% sucrose. Each set of data (from either 4 or 12 technical replicate cultures of the same strain) is labeled with the pyrophosphatase enzyme introduced into that strain, including the previously described enzyme TalVeTPP. Data are represented as boxplots, with values shown relative to titers from a TalVeTPP control strain.

### DETAILED DESCRIPTION OF THE INVENTION

### Definitions

As used herein, the singular forms “a,” “an,” and “the” include plural reference unless the context clearly dictates otherwise.

As used herein, the term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “capable of producing” refers to a host cell which is genetically modified to include the enzymes necessary for the production of a given compound in accordance with a biochemical pathway that produces the compound. For example, a cell (e.g., a yeast cell) “capable of producing” an isoprenoid compound is one that contains the enzymes necessary for production of the isoprenoid compound according to the isoprenoid biosynthetic pathway.

As used herein, the term “exogenous” refers to a substance or compound that originated outside an organism or cell. The exogenous substance or compound can retain its normal function or activity when introduced into an organism or host cell described herein.

As used herein, the term “fermentation composition” refers to a composition which contains genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refer to a set of at least two different coding sequences, where the coding sequences encode enzymes that catalyze different parts of a synthetic pathway to form a desired product (e.g., an isoprenoid). In a genetic pathway a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “genetic switch” refers to one or more genetic elements that allow controlled expression of enzymes, e.g., enzymes that catalyze the reactions of isoprenoid biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “heterologous” refers to what is not normally found in nature. The term “heterologous compound” refers to the production of a compound by a cell that does not normally produce the compound, or to the production of a compound at a level not normally produced by the cell. For example, an isoprenoid can be a heterologous compound.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture that contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the terms “isoprenoid”, “isoprenoid compound,” “isoprenoid product,” “terpene,” “terpene compound,” “terpenoid,” and “terpenoid compound” are used interchangeably. They refer to compounds that are capable of being derived from IPP.

As used herein, the term “medium” refers to culture medium and/or fermentation medium.

As used herein, the terms “modified,” “genetically modified,” “recombinant,” and “engineered,” when used to describe a host cell described herein, refer to host cells or organisms that do not exist in nature, host cells or organisms that express compounds, nucleic acids, or proteins at levels that are not expressed by naturally occurring cells or organisms, or host cells or organisms into which a gene or DNA sequence is introduced, regardless of whether the same or similar gene or DNA sequence is already present in the host cell or organism. Thus, a genetically modified host cell can comprise, for example, a DNA sequence from another species or can be a DNA sequence that originated from or is present in the same species as the host, but has been incorporated into a host by recombinant methods to form a genetically modified host cell. It will be appreciated that a recombinant gene that is introduced into a host can be identical to a DNA sequence that is normally present in the host being transformed, and is introduced to provide one or more additional copies of the DNA sequence to thereby permit overexpression or modified expression of the gene product of the DNA sequence.

As used herein, the term “naturally occurring” as applied to a nucleic acid, an enzyme, a cell, or an organism, refers to a nucleic acid, enzyme, cell, or organism that is found in nature. For example, a polypeptide or polynucleotide sequence that is present in an organism that can be isolated from a source in nature and that has not been intentionally modified by a human in the laboratory is naturally occurring. As used herein, the term “non-naturally occurring” means what is not found in nature but is created by human intervention.

As used herein, the phrase “operably linked” refers to a functional linkage between nucleic acid sequences such that the linked promoter and/or regulatory region functionally controls expression of the coding sequence.

As used herein, the terms “overlay,” “oil,” “overlay oil,” or “oil overlay” refer to a biologically compatible hydrophobic, lipophilic, carbon-containing substance including but not limited to geologically-derived crude oil, distillate fractions of geologically-derived crude oil, vegetable oil, algal oil, microbial lipids, or synthetic oils. The oil is neither itself toxic to a biological molecule, a cell, a tissue, or a subject, nor does it degrade (if the oil degrades) at a rate that produces byproducts at toxic concentrations to a biological molecule, a cell, a tissue or a subject.

As used here, “percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as CLUSTAL, BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages; positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally derived nucleic acid that is capable of activating, increasing or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

As used herein, the term “pyrophosphate” is used interchangeably herein with “diphosphate.”

As used herein, the term “pyrophosphatase” refers to an enzyme having pyrophosphatase activity, i.e., cleaves pyrophosphate from a substrate. For example, TalVeTPP (SEQ ID NO: 19), a phosphatase enzyme from the fungal species Talaromyces verruculosus, has been shown to convert copalyl-pyrophosphate into E-copalol when expressed in the yeast S. cerevisiae or in the bacterium E. coli, and therefore can be a pyrophosphatase.

The term “yield” refers to production of a compound by a host cell, expressed as the amount of compound produced per amount of carbon source consumed by the host cell, by weight.

### High Efficiency Production of Isoprenoid Compounds

In an aspect, the disclosure features genetically modified host cell capable of producing E-copalol, wherein the genetically modified host cell comprises one or more heterologous nucleic acids that each, independently, encodes an enzyme comprising an amino acid sequence that is at least 90% identical to the amino acid sequence of SEQ ID NOS. 3, 6, 9, 12, 15, or 18. In some embodiments, the enzyme comprises the amino acid sequence of SEQ ID NOS. 3, 6, 9, 12, 15, or 18.

In another aspect, the disclosure features a genetically modified host cell capable of producing E-copalol, wherein the genetically modified host cell comprises one or more heterologous nucleic acids that each, independently, encodes an enzyme capable of converting copalyl-diphosphate (CPP) to E-copalol. In some embodiments, the enzyme capable of converting CPP to E-copalol is a pyrophosphatase.

In some embodiments, the genetically modified host cell further comprises one or more heterologous nucleic acids that each, independently, encodes one or more enzymes of a pathway for making E-copalol. In some embodiments, the genetically modified host cell further comprises one or more heterologous nucleic acids that each, independently, encodes an enzyme comprising the amino acid sequence of SEQ ID NO. 24, SEQ ID NO. 39, SEQ ID NO. 42, SEQ ID NO. 43, or SEQ ID NO. 45. In some embodiments, the genetically modified host cell further comprises one or more heterologous nucleic acids that each, independently, encodes an enzyme capable of converting one or more IPP, DMAPP, GPP, FPP, or GGPP into GPP, FPP, GGPP, or CPP. In some embodiments, the genetically modified host cell further comprises a CPP synthase, an Erg20, a GPP synthase, or a GGPP synthase.

In some embodiments, expression of one or more of the enzymes disclosed herein is under the control of a single transcriptional regulator. In some embodiments, expression of one or more of the enzymes disclosed herein is under the control of multiple transcriptional regulators.

In some embodiments, the genetically modified host cell is a yeast cell or a yeast strain. In some embodiments, the yeast cell or the yeast strain is Saccharomyces cerevisiae.

In an aspect, the disclosure provides for a fermentation composition, comprising a genetically modified host cell disclosed herein, optionally an overlay, and E-copalol produced by the genetically modified host cell.

In another aspect, the disclosure provides for a method for producing E-copalol, comprising culturing a genetically modified host cell disclosed herein in a medium with a carbon source under conditions suitable for making E-copalol, optionally providing an overlay, and recovering E-copalol from the genetically modified host cell, the overlay, or the medium.

In yet another aspect, the disclosure provides for a non-naturally occurring enzyme capable of converting CPP to E-copalol comprising an amino acid sequence that is at least 90% identical to the amino acid sequence of SEQ ID NOS. 3, 6, 9, 12, 15, or 18. In an embodiment, the non-naturally occurring enzyme comprises the amino acid sequence of SEQ ID NOS. 3, 6, 9, 12, 15, or 18.

### MEV Pathway

In general, the mevalonate pathway comprises six steps. In the first step, two molecules of acetyl-coenzyme A are enzymatically combined to form acetoacetyl-CoA. An enzyme known to catalyze this step is, for example, acetyl-CoA thiolase (also known as acetyl-CoA acetyltransferase).

In the second step of the MEV pathway, acetoacetyl-CoA is enzymatically condensed with another molecule of acetyl-CoA to form 3-hydroxy-3-methylglutaryl-CoA (HMG-CoA). An enzyme known to catalyze this step is, for example, HMG-CoA synthase.

In the third step, HMG-CoA is enzymatically converted to mevalonate. An enzyme known to catalyze this step is, for example, HMG-CoA reductase.

In the fourth step, mevalonate is enzymatically phosphorylated to form mevalonate 5-phosphate. An enzyme known to catalyze this step is, for example, mevalonate kinase.

In the fifth step, a second phosphate group is enzymatically added to mevalonate 5-phosphate to form mevalonate 5-pyrophosphate. An enzyme known to catalyze this step is, for example, phosphomevalonate kinase.

In the sixth step, mevalonate 5-pyrophosphate is enzymatically converted into IPP. An enzyme known to catalyze this step is, for example, mevalonate pyrophosphate decarboxylase.

If IPP is to be converted to DMAPP, then a seventh step is required. An enzyme known to catalyze this step is, for example, IPP isomerase. If the conversion to DMAPP is required, an increased expression of IPP isomerase ensures that the conversion of IPP into DMAPP does not represent a rate-limiting step in the overall pathway.

### DXP Pathway

In general, the DXP pathway comprises seven steps. In the first step, pyruvate is condensed with D-glyceraldehyde 3-phosphate to make 1-deoxy-D-xylulose-5-phosphate. An enzyme known to catalyze this step is, for example, 1-deoxy-D-xylulose-5-phosphate synthase.

In the second step, 1-deoxy-D-xylulose-5-phosphate is converted to 2C-methyl-D-erythritol-4-phosphate. An enzyme known to catalyze this step is, for example, 1-deoxy-D-xylulose-5-phosphate reductoisomerase.

In the third step, 2C-methyl-D-erythritol-4-phosphate is converted to 4-diphosphocytidyl-2C-methyl-D-erythritol. An enzyme known to catalyze this step is, for example, 4-diphosphocytidyl-2C-methyl-D-erythritol synthase.

In the fourth step, 4-diphosphocytidyl-2C-methyl-D-erythritol is converted to 4-diphosphocytidyl-2C-methyl-D-erythritol-2-phosphate. An enzyme known to catalyze this step is, for example, 4-diphosphocytidyl-2C-methyl-D-erythritol kinase.

In the fifth step, 4-diphosphocytidyl-2C-methyl-D-erythritol-2-phosphate is converted to 2C-methyl-D-erythritol 2, 4-cyclodiphosphate. An enzyme known to catalyze this step is, for example, 2C-methyl-D-erythritol 2, 4-cyclodiphosphate synthase.

In the sixth step, 2C-methyl-D-erythritol 2, 4-cyclodiphosphate is converted to 1-hydroxy-2-methyl-2-(E)-butenyl-4-diphosphate. An enzyme known to catalyze this step is, for example, 1-hydroxy-2-methyl-2-(E)-butenyl-4-diphosphate synthase.

In the seventh step, 1-hydroxy-2-methyl-2-(E)-butenyl-4-diphosphate is converted into either IPP or its isomer, DMAPP. An enzyme known to catalyze this step is, for example, isopentyl/dimethylallyl diphosphate synthase.

In some embodiments, “cross talk” (or interference) between the host cell's own metabolic processes and those processes involved with the production of IPP as provided herein are minimized or eliminated entirely. For example, cross talk is minimized or eliminated entirely when the host microorganism relies exclusively on the DXP pathway for synthesizing IPP, and a MEV pathway is introduced to provide additional IPP. Such a host organisms would not be equipped to alter the expression of the MEV pathway enzymes or process the intermediates associated with the MEV pathway. Organisms that rely exclusively or predominately on the DXP pathway include, for example, Escherichia coli.

In some embodiments, the host cell produces IPP via the MEV pathway, either exclusively or in combination with the DXP pathway. In other embodiments, a host's DXP pathway is functionally disabled so that the host cell produces IPP exclusively through a heterologously introduced MEV pathway. The DXP pathway can be functionally disabled by disabling gene expression or inactivating the function of one or more of the DXP pathway enzymes.

### E-Copalol Pathway

Several routes to E-copalol are possible. One pathway, from IPP and DMAPP to E-copalol, comprises two steps. In the first step, three IPP and one DMAPP are converted to copalyl-pyrophosphate (CPP). Enzymes known to catalyze this step are, for example, chimeric diterpene synthases from Penicillium species. Illustrative examples of nucleotide sequences include but are not limited to SEQ ID NOS: 22 and 23. In the second step, CPP is converted to E-copalol. An enzyme known to catalyze this step is, for example, a CPP pyrophosphatase. Illustrative examples of nucleotide sequences include but are not limited to SEQ ID NOS. 1, 2, 4, 5, 7, 8, 10, 11, 13, 14, 16, 17, 19, and 20.

In another route, two IPP and one DMAPP are converted to FPP. An enzyme known to catalyze this step is, for example, S. cerevisiae Erg20. An illustrative example of a nucleotide sequence includes but is not limited to SEQ ID NO: 44. One IPP and one FPP are then converted to CPP. Enzymes known to catalyze this step are, for example, chimeric diterpene synthases from Penicillium species. Illustrative examples of nucleotide sequences include but are not limited to SEQ ID NOS: 22 and 23. CPP is then converted to E-copalol. An enzyme known to catalyze this step is, for example, a CPP pyrophosphatase. Illustrative examples of nucleotide sequences include but are not limited to SEQ ID NOS. 1, 2, 4, 5, 7, 8, 10, 11, 13, 14, 16, 17, 19, and 20.

Another route involves conversion of two IPP and one DMAPP to form FPP. An enzyme known to catalyze this step is, for example, S. cerevisiae Erg20. An illustrative example of a nucleotide sequence includes but is not limited to SEQ ID NO: 44. One FPP and one IPP are then converted to GGPP. An enzyme known to catalyze this step is, for example, a GGPP synthase. Illustrative examples of nucleotide sequences include but are not limited to SEQ ID NOS: 37 and 38. GGPP is then converted to CPP. An enzyme known to catalyze this step is, for example, a CPP synthase. Illustrative examples of nucleotide sequences include but are not limited to SEQ ID NOS: 40 and 41. Finally, CPP is converted to E-copalol. An enzyme known to catalyze this step is, for example, a CPP pyrophosphatase. Illustrative examples of nucleotide sequences include but are not limited to SEQ ID NOS. 1, 2, 4, 5, 7, 8, 10, 11, 13, 14, 16, 17, 19, and 20.

### Methods of Making Genetically Modified Host Cells

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons more frequently. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon.

Those of skill in the art will recognize that, due to the degenerate nature of the genetic code, a variety of DNA molecules differing in their nucleotide sequences can be used to encode a given enzyme of the disclosure. Any one of the polypeptide sequences disclosed herein may be encoded by DNA molecules of any sequence that encode the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) can be considered homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used for comparing a molecule sequence to a database containing a large number of sequences from different organisms is the computer algorithm BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed in the host cell. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. stipitis, Torulaspora pretoriensis, Issatchenkia orientalis, Schizosaccharomyces spp., including S. pombe, Cryptococcus spp., Aspergillus spp., including A. leporis, A. alliaceus, A. brasiliensis, and A. wentii, Neurospora spp., Ustilago spp., Talaromyces spp., including T. amestolkiae, Parastagonospora spp., including P. nodorum, Phaeosphaeria spp., including P. poagena, Stagonospora spp., Aureobasidium spp., including A. pullulans, Lepidopterella spp., including L. palustris, or Rhinocladiella spp., including R. mackenziei. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous kinase genes, proteins, or enzymes, techniques may include, but are not limited to, cloning a gene by PCR using primers based on a published sequence of a kinase gene/enzyme or by degenerate PCR using degenerate primers designed to amplify a conserved region among kinase genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, JGI Phyzome v12.1, BLAST, NCBI RefSeq, UniProt KB, or MetaCYC Protein annotations in the UniProt Knowledgebase may also be used to identify enzymes which have a similar function in addition to the National Center for Biotechnology Information RefSeq database. The candidate gene or enzyme may be identified within the above-mentioned databases in accordance with the teachings herein.

### Genetically Modified Host Cells

In one aspect, provided herein are host cells comprising at least one enzyme of the isoprenoid biosynthetic pathway. In some embodiments, the isoprenoid biosynthetic pathway contains a genetic regulatory element, such as a nucleic acid sequence, that is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate production of an isoprenoid compound and provide a carbon source for growth of the host cell. In some embodiments, the exogenous agent is galactose. In some embodiments, the exogenous agent is maltose.

In some embodiments, the genetic regulatory element is a nucleic acid sequence, such as a promoter.

In some embodiments, the genetic regulatory element is a galactose-responsive promoter. In some embodiments, galactose positively regulates expression of the isoprenoid biosynthetic pathway, thereby increasing production of the isoprenoid compound. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, the host cell lacks the gall gene and is unable to metabolize galactose, but galactose can still induce galactose-regulated genes.

TABLE A
Exemplary GAL Promoter Sequences

| Promoter | Sequence |
|---|---|
| pGAL1 | SEQ ID NO: 25 |
| pGAL10 | SEQ ID NO: 26 |
| pGAL2 | SEQ ID NO: 27 |
| pGAL3 | SEQ ID NO: 28 |
| pGAL7 | SEQ ID NO: 29 |
| pGAL4 | SEQ ID NO: 30 |

In some embodiments, the galactose regulation system used to control expression of one or more enzymes of the isoprenoid biosynthetic pathway is re-configured such that it is no longer induced by the presence of galactose. Instead, the gene of interest will be expressed unless repressors, which may be maltose in some strains, are present in the medium.

In some embodiments, the genetic regulatory element is a maltose-responsive promoter. In some embodiments, maltose negatively regulates expression of the isoprenoid biosynthetic pathway, thereby decreasing production of the isoprenoid compound. In some embodiments, the maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium.

TABLE B
Exemplary MAL Promoter Sequences

| Promoter | Sequence |
|---|---|
| pMAL1 | SEQ ID NO: 31 |
| pMAL2 | SEQ ID NO: 32 |
| pMAL11 | SEQ ID NO: 33 |
| pMAL12 | SEQ ID NO: 34 |
| pMAL31 | SEQ ID NO: 35 |
| pMAL32 | SEQ ID NO: 36 |

In some embodiments, the heterologous genetic pathway is regulated by a combination of the maltose and galactose regulons.

In some embodiments, the recombinant host cell does not contain, or expresses a very low level of (for example, an undetectable amount), a precursor required to make the isoprenoid compound. In some embodiments, the precursor is a substrate of an enzyme in the isoprenoid biosynthetic pathway.

### Yeast Strains

In some embodiments, yeast strains useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK. In some embodiments, the strain of Saccharomyces cerevisiae is CEN.PK2.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### Transformation of Genetically Modified Host Cells

In another aspect, provided are methods of making the modified host cells described herein. In some embodiments, the methods include transforming a host cell with the heterologous nucleic acid constructs described herein which encode the proteins expressed by a heterologous genetic pathway described herein.

### Methods for Producing an Isoprenoid Compound

In another aspect, methods for producing an isoprenoid compound are described herein. In some embodiments, the method decreases expression of the isoprenoid compound. In some embodiments, the method includes culturing a host cell comprising at least one enzyme of the isoprenoid biosynthetic pathway described herein in a medium comprising an exogenous agent, wherein the exogenous agent decreases the expression of the isoprenoid compound. In some embodiments, the exogenous agent is maltose. In some embodiments, the method results in less than 0.001 mg/L of an isoprenoid compound or a precursor thereof.

In some embodiments, the method is for decreasing expression of an isoprenoid compound or precursor thereof. In some embodiments, the method includes culturing a host cell comprising one or more enzymes of the isoprenoid biosynthetic pathway described herein in a medium comprising an exogenous agent, wherein the exogenous agent decreases the expression of the isoprenoid compound. In some embodiments, the exogenous agent is maltose. In some embodiments, the exogenous agent is maltose. In some embodiments, the method results in the production of less than 0.001 mg/L of an isoprenoid compound or a precursor thereof.

In some embodiments, the method increases the expression of an isoprenoid compound. In some embodiments, the method includes culturing a host cell comprising one or more enzymes of the isoprenoid biosynthetic pathway described herein in a medium comprising the exogenous agent, wherein the exogenous agent increases expression of the isoprenoid compound. In some embodiments, the exogenous agent is galactose. In some embodiments, the method further includes culturing the host cell with the precursor or substrate required to make the isoprenoid compound.

In some embodiments, the method increases the expression of an isoprenoid compound or precursor thereof. In some embodiments, the method includes culturing a host cell comprising a heterologous isoprenoid compound described herein in a medium comprising an exogenous agent, wherein the exogenous agent increases the expression of the isoprenoid compound or a precursor thereof. In some embodiments, the exogenous agent is galactose. In some embodiments, the method further includes culturing the host cell with a precursor or substrate required to make the isoprenoid compound or precursor thereof. In some embodiments, the combination of the exogenous agent and the precursor or substrate required to make the isoprenoid compound or precursor thereof produces a higher yield of the isoprenoid compound than the exogenous agent alone.

### Culture and Fermentation Methods

Materials and methods for the maintenance and growth of microbial cultures are well known to those skilled in the art of microbiology or fermentation science. Consideration must be given to appropriate culture medium, pH, temperature, and requirements for aerobic, microaerobic, or anaerobic conditions, depending on the specific requirements of the host cell, the fermentation, and the process.

The methods of producing isoprenoid compounds provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, a complex feedstock, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol. Non-limiting examples of a complex feedstock include cane syrup.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of isoprenoid compounds may also occur in these culture conditions, but at undetectable levels (with detection limits being about <0.1 g/1). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and sometimes less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution of metal salts that, for convenience, can be prepared separately from the rest of the culture medium, with individual components of the stock solution added, for example, at concentrations ranging from 0.3 g/L to 6 g/L. Typically, the amount of such a trace metals solution added to the culture medium is greater than about 1 mL/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as biotin, calcium pantothenate, inositol, p-aminobenzoic acid, nicotinic acid, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components can be maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the fermenter and maintained in the range of from about 1 g/L to about 100 g/L, or in the range of from about 2 g/L to about 50 g/L, or in the range of from about 5 g/L to about 20 g/L. Alternatively, the glucose concentration in the culture medium is maintained below detection limits. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Yeast Transformation Methods

Each DNA construct was integrated into Saccharomyces cerevisiae (CEN.PK2) with standard molecular biology techniques in an optimized lithium acetate (LiAc) transformation. Briefly, cells were grown overnight in standard liquid culture medium at 30° C. with shaking (200 rpm), diluted to an OD₆₀₀ of 0.1 in fresh medium, and grown to an OD₆₀₀ of 0.6-0.8. For each transformation, 5 mL of culture were harvested by centrifugation, washed in 5 mL of sterile water, spun down again, resuspended in 1 mL of 100 mM LiAc, and transferred to a microcentrifuge tube. Cells were spun down (13,000×g) for 30 seconds, the supernatant was removed, and the cells were resuspended in a transformation mix of 240 μL 50% PEG, 36 μL 1 M LiAc, 10 μL boiled salmon sperm DNA, and 74 μL of donor DNA (˜1 μg). Following a heat shock at 42° C. for 40 minutes, cells were centrifuged and suspended in liquid culture medium for overnight recovery at 30° C. with shaking (200 rpm) before plating on solid agar selective medium. DNA integration was confirmed by yeast colony PCR with primers specific to the integrations.

### Example 2: Construction of Yeast Strain to Identify and Rank Novel Pyrophosphatase Enzymes that Convert Copalyl-Pyrophosphate (CPP) into E-Copalol

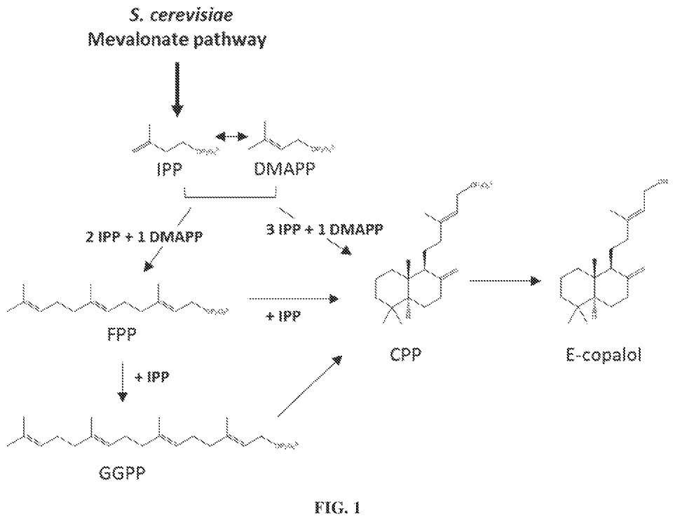

A subset of enzymes from the larger family of protein tyrosine phosphatases have been shown to cleave the terpenyl-diphosphate linkage in CPP to generate E-copalol. For example, TalVeTPP (SEQ ID NO: 2), a phosphatase enzyme from the fungal species Talaromyces verruculosus, converts CPP into E-copalol when expressed in the yeast S. cerevisiae. FIG. 1 shows exemplary biosynthetic pathways from the native S. cerevisiae metabolites IPP and DMAPP to E-copalol through the intermediates FPP, GGPP, and CPP.

CPP production in a S. cerevisiae base strain (CEN.PK2) was enabled by integrating and expressing a codon-optimized version (SEQ ID NO: 23) of the heterologous CPP synthase gene PvCPS under control of a strong S. cerevisiae promoter. In addition to PvCPS, one of various candidate CPP pyrophosphatases was introduced into strains to enable possible conversion of CPP into E-copalol. Candidate pyrophosphatases were identified from sequence databases based on similarity to known CPP pyrophosphatases such as TalVeTPP. DNA sequences were codon-optimized for expression in S. cerevisiae, and integrated into the genome under control of a strong S. cerevisiae promoter. To generate an isogenic control strain, a codon-optimized gene expressing TalVeTPP (SEQ ID NO: 20) was integrated into the genome along with PvCPS under control of the same strong promoter.

### Example 3: Yeast Culturing Conditions in 96-Well Plates

Yeast were inoculated into 96-well microtiter plates containing 120 μL per well Bird Seed Media (100 ml/L Bird Batch (potassium phosphate 80 g/L, ammonium Sulfate 150 g/L, magnesium sulfate 61.5 g/L), 5 ml/L Trace Metal Solution (0.5M EDTA 160 mL/L, zinc sulfate heptahydrate 11.5 g/L, copper sulfate 0.64 g/L, manganese(II) chloride 0.64 g/L, cobalt(II) chloride hexahydrate 0.94 g/L, sodium molybdate 0.96 g/L, iron(II) sulfate 5.6 g/L, calcium chloride dihydrate 5.8 g/L), 12 ml/L Birds Vitamins 2.0 (biotin 0.05 g/L, p-aminobenzoic acid 0.2 g/L, calcium pantothenate 1 g/L, nicotinic acid 1 g/L, myoinositol 25 g/L, thiamine HCl 1 g/L, pyridoxine HCl 1 g/L), and succinic acid 6 g/L; pH 5), with 4% sucrose, and a hydrophobic isopropyl myristate overlay added at 25% of aqueous volume. Microtiter plates were sealed with gas-permeable membranes and cultured at 30° C. in a high-capacity microtiter plate incubator shaking at 1000 rpm and 80% humidity for 3 days, by which time cultures had reached carbon exhaustion.

### Example 4: Assessment of CPP Pyrophosphatase Performance

To quantify the amount of E-copalol produced by cultures of strains that contain different candidate CPP pyrophosphatases, E-copalol was measured by gas chromatography (GC). The different candidate pyrophosphatases were ranked based on E-copalol titers of the cultures in which they were expressed. To assess titers, cultures from 96-well plates were extracted with 10 volumes of ethyl acetate (relative to aqueous volume) by shaking at 1000 rpm for 30 seconds at room temperature, and extractant was separated by centrifugation (2000 rpm for 5 minutes) and analyzed on an Agilent 7890A with flame ionization detection (GC-FID) along with analytical standards. The following ramped temperature program with constant flow at 1.4 mL/min was used for analysis:

| | |
|---|---|
| Initial Temp (C.) | 160 |
| Initial Hold (min) | 0.16 |
| Rate 1 (C./min) | 100 |
| Temp 1 (C.) | 193 |
| Hold Time (min) | 2 |
| Rate 2 (C./min) | 60 |
| Temp 2 (C.) | 250 |
| Hold Time (min) | 2 |
| Rate 3 (C./min) | 100 |
| Temp 3 (C.) | 320 |
| Hold Time (min) | 1.86 |
| Run Time (min) | 9 |

### Example 5: Six Novel Enzymes have Improved Activity in Conversion of CPP to E-Copalol Relative to TalVeTPP

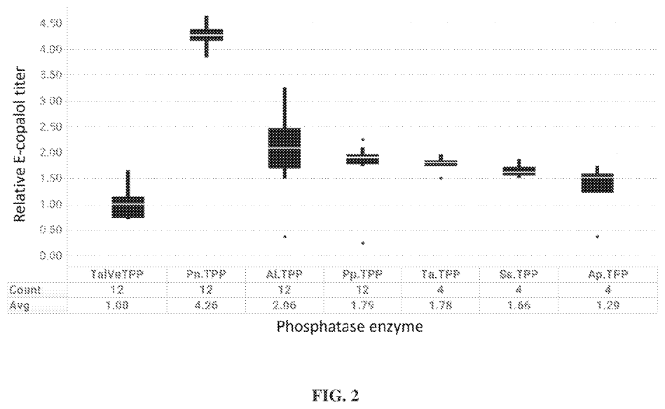

Native enzymes from six different fungal species demonstrated improved activity in conversion of CPP to E-copalol relative to TalVeTPP when expressed in S. cerevisiae CPP-producing strains as described in Example 2. FIG. 2 illustrates the performance of strains that were engineered to express either TalVeTPP or one of these six new enzymes when they were grown in plate cultures as described in Example 3, and assessed based on measurement of E-copalol titers after culturing as described in Example 4. Table 1 summarizes the average (mean) performance of all these strains, with improvements in E-copalol titers ranging from 30% to 325% over the TalVeTPP control strain.

TABLE 1
Summary of relative E-Copalol titers from data shown in
FIG. 2. CPP-producing strains listed each contain one
copy of the pyrophosphatase enzyme shown in the table.

| | | | Relative E- |
|---|---|---|---|
| Phosphatase | SEQ ID | | copalol |
| enzyme | NO. | Source organism | produced |
| TalVeTPP | 21 | Talaromyces verruculosus | 1.00 |
| (control) | | | |
| Pn.TPP | 3 | Parastagonospora nodorum SN15 | 4.26 |
| Al.TPP | 6 | Aspergillus leporis | 2.06 |
| Pp.TPP | 9 | Phaeosphaeria poagena | 1.79 |
| | | MPI-PUGE-AT-0046c | |
| Ta.TPP | 12 | Talaromyces amestolkiae | 1.78 |
| Ss.TPP | 15 | Stagonospora sp. SRC1lsM3a | 1.66 |
| Ap.TPP | 18 | Aureobasidium pullulans | 1.29 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

All publications, patents, and patent applications cited in this specification are herein incorporated by reference as if each individual publication or patent application were specifically and individually indicated to be incorporated by reference.

SEQUENCE APPENDIX

SEQ ID NO: 1; Pn. TPP wild-type cDNA
ATGTCAACCACAAAAGAAGACCAACCTCTCCCCACTCCCCCCTTCCACATCGTCCCCAACATCA
ACAACCTTCGAGACGCCGCCCTCTTCCCCCTAACCACCCCCTCCGGCTCAATCCGCCCCAAAAT
CCTCTTCCGCTCCGCCGACGTCTCCAAACTCCCCCTCAGCGGCTGGCAAGCCCTCCATTCCCTG
GGTATCACCCATGTCTTCGACCTACGCAGCGCGCCCGAAGTCGGATTCCGCGACTCGGACACCT
CCAAGCCAGAATGGGTCAGCGCCATGACCTCCGTCGGCATCAAGCGCACGTGGTGCCCCGTCTT
CACTGAAGCAGACTACTCCCCCGAGGGCCTCGCCAAGCGCTACGTCAAGTACATGGACGAAGAC
GTGGCGGGCTTTGTATCCGCGTACCACGACATCCTTCTCGACGGCGGCGCAGCATACACCACTA
TCCTGCGCCACCTAATTGATCATCCCGGCGAGGGAGTGTTGATTCACTGCACGGCGGGCAAAGA
CCGCACGGGCATCTTCTTCGGCTTGCTATTCGCCTACCTGGGCGTGGACAGCCAAGTCATTGCT
GAGGAGTACAACCTGACGGAACTCGGGCTGACGCATGTGCGCGAAGAAGTCGTCGCGAGACTGT
TGCAGTCCCCGGCGTTTAAGAATTACATCGCGACCAAGGCGACCGGAAAGCAGCTTAGCGCCGA
GGAGATTGGGAAGTTGATTGCGGACGACAAGGCGGGGAAGCAGAGTGGTGTCGAGGAGACATTG
GATCCGGAGGCGAGGAAGCAGGGCAGGGAGGCTGCGCTGAGGATGGTGGGGGCGAAGAAGGAGA
CGATGGTTAAGGCGTTGGAGATGCTGGAGAGGGATTTTGGGGGCGCGGAGAAGTATCTGCGCGA
GAAGTGTGGTCTGGGAGATGGGGATTTGGAGAAGTTGAGGAGGAATTTGGTGGTGCGTGAGGAG
GCGTGA
SEQ ID NO: 2; Pn. TPP optimized cDNA
ATGTCTACAACTAAAGAAGATCAACCATTGCCCACGCCTCCATTCCACATCGTTCCAAACATTA
ACAACTTGAGAGACGCTGCCTTATTCCCATTGACCACTCCATCTGGTTCCATCAGACCAAAGAT
TTTGTTCAGATCTGCTGACGTTTCCAAATTACCTTTGTCTGGTTGGCAAGCCTTGCACTCTTTG
GGTATTACTCACGTCTTCGATTTAAGATCTGCCCCAGAAGTCGGTTTTAGAGATTCTGATACCT
CCAAGCCAGAATGGGTTTCTGCTATGACCTCCGTTGGTATCAAGAGAACCTGGTGTCCTGTTTT
CACTGAAGCCGATTACTCTCCTGAAGGTTTGGCTAAGCGTTACGTCAAGTACATGGATGAGGAT
GTTGCTGGTTTCGTTTCTGCTTATCATGACATTTTGTTGGACGGTGGTGCTGCTTATACTACCA
TTTTGAGACACTTAATCGATCATCCAGGTGAAGGTGTCTTGATTCACTGTACTGCTGGTAAGGA
TCGTACTGGTATCTTCTTCGGTTTGTTGTTCGCTTACTTGGGTGTTGATTCCCAAGTTATTGCC
GAAGAATATAACTTGACTGAATTGGGTTTGACTCACGTTAGAGAAGAAGTTGTCGCTAGATTGT
TGCAATCCCCAGCTTTCAAGAACTACATTGCCACTAAGGCTACCGGTAAACAATTGTCTGCTGA
AGAAATCGGTAAGTTGATCGCTGACGATAAGGCTGGTAAACAATCCGGTGTTGAAGAAACTTTG
GATCCAGAGGCTAGAAAACAAGGTAGAGAAGCTGCTTTGAGAATGGTTGGTGCTAAAAAAGAAA
CTATGGTTAAGGCTTTGGAGATGTTGGAAAGAGATTTTGGTGGTGCTGAAAAGTACTTGAGAGA
AAAATGTGGTTTGGGTGACGGTGACTTGGAAAAGTTGCGTAGAAACTTGGTTGTCAGAGAAGAA
GCTTAA
SEQ ID NO: 3; Pn. TPP amino acid sequence
MSTTKEDQPLPTPPFHIVPNINNLRDAALFPLTTPSGSIRPKILFRSADVSKLPLSGWQALHSL
GITHVFDLRSAPEVGFRDSDTSKPEWVSAMTSVGIKRTWCPVFTEADYSPEGLAKRYVKYMDED
VAGFVSAYHDILLDGGAAYTTILRHLIDHPGEGVLIHCTAGKDRTGIFFGLLFAYLGVDSQVIA
EEYNLTELGLTHVREEVVARLLQSPAFKNYIATKATGKQLSAEEIGKLIADDKAGKQSGVEETL
DPEARKQGREAALRMVGAKKETMVKALEMLERDEGGAEKYLREKCGLGDGDLEKLRRNLVVREE
A
SEQ ID NO: 4; Al. TPP wild-type cDNA
ATGAGTCTTTCTCTTCCTTTTATTCATGTGGAGGGGGTGAGCAACTTTAGGAGCTTGGGGGAT
ACCCAGTCGCCACATGTACACCTGGCAAAAAACCACTTACCACCCGTCAGCATTTCTTCTACCG
CAGTGCGGATCTGGTCAAGATAACGGAATCCGGCCGAGCTACGATGCAATCGCTGGGGGTTACA
AGCGTGTATGATCTGCGCTCCGTTGGCGAAGGTCAAAGGGCACAGGCCGCACACAACCAGGCAG
GCATCACCACAAGATTCAATAGCATAGCTCTAGGGCCCGACATCAAGGTGCACCCAACTCCCAT
TTTTGCCCACGAGGACTGGAGCCCGGAAGCAGTAGGCGAGCGGTACCTTCGGTATGCCCAGGAG
CACGCCCTATCCGGCTCGGGCTATGCAGAGGTCTACCGAGACATGCTAGAACGGGGACACGAGG
CCATCCGCGTCGTCCTTCTGCACGTACGAGACCATCCGACCGAGCCCTTTCTCTGTCATTGTAG
CGCAGGCAAGGACCGCACGGGTGTCGTCGTCGCCGTGTTGCTAAAGCTGGCCGGATGCGACGAT
GAGGTGGTGGCACGCGAATATGCGCTCACGGAGCTGGGACTGGCGGCCCGTAAGGAGTTTATCG
TGCAGTACCTCTTGCGGAAACCAGAGGTGAAAGGTTCACGGGTGCTCGCCGAGCGAGTAGCCAG
TGCGTCTTATTCCAACATGTGGGAGACCCTGCAGATGGTCCGAAGCAAATACGGGAGCATGGCT
GGTTATGTGATGAAGTTCTGTAGTCTTACCGAGTCTGATCTGGACAGCATCCAACAGAATCTGA
CCTGCTCGGATGCGCCGAGTCCGTGGGCCGTATCGATATAG
SEQ ID NO: 5; Al. TPP optimized cDNA
ATGAGCCTTTCCCTGCCTTTTATTCATGTCGAGGGTGTCTCTAATTTCAGATCCTTAGGTGGTT
ACCCTGTTGCCACTTGTACTCCAGGTAAGAAGCCATTAACTACCAGACAACACTTCTTCTACAG
ATCTGCTGATTTAGTCAAGATTACTGAATCTGGTAGAGCTACCATGCAATCTTTGGGTGTTACT
TCTGTTTACGACTTGAGATCCGTCGGTGAAGGTCAACGTGCCCAAGCTGCTCATAACCAAGCCG
GTATTACTACCAGATTCAATTCCATTGCTTTAGGTCCAGACATTAAGGTTCACCCTACTCCAAT
TTTCGCTCATGAAGATTGGTCCCCTGAAGCTGTTGGTGAAAGATATTTAAGATACGCTCAAGAA
CATGCTTTGTCCGGTTCTGGTTACGCTGAAGTTTACAGAGATATGTTGGAAAGAGGTCATGAAG
CCATCAGAGTTGTTTTGTTGCACGTCAGAGATCACCCAACCGAACCATTTTTGTGTCATTGTTC
TGCTGGTAAGGATAGAACCGGTGTTGTCGTCGCCGTTTTGTTGAAGTTAGCTGGTTGTGATGAC
GAAGTTGTTGCTAGAGAATATGCTTTAACTGAATTGGGTTTGGCTGCTAGAAAGGAATTCATTG
TTCAATACTTGTTGAGAAAGCCTGAAGTCAAAGGTTCTAGAGTCTTGGCCGAAAGAGTTGCCTC
CGCCTCCTACTCTAACATGTGGGAAACTTTGCAAATGGTTAGATCTAAATACGGTTCTATGGCT
GGTTACGTTATGAAGTTCTGTTCTTTGACTGAATCTGATTTGGACTCTATTCAACAAAACTTGA
CCTGTTCCGATGCTCCATCTCCTTGGGCTGTCTCCATTTAG
SEQ ID NO: 6; Al. TPP amino acid sequence
MSLSLPFIHVEGVSNFRSLGGYPVATCTPGKKPLITROHFFYRSADLVKITESGRATMQSLGVT
SVYDLRSVGEGQRAQAAHNQAGITTRFNSIALGPDIKVHPTPIFAHEDWSPEAVGERYLRYAQE
HALSGSGYAEVYRDMLERGHEAIRVVLLHVRDHPTEPFLCHCSAGKDRTGVVVAVLLKLAGCDD
EVVAREYALTELGLAARKEFIVQYLLRKPEVKGSRVLAERVASASYSNMWETLQMVRSKYGSMA
GYVMKFCSLTESDLDSIQQNLTCSDAPSPWAVSI
SEQ ID NO: 7; Pp. TPP wild-type cDNA
ATGTCGAGCCCCCAAGCCCTCCCCTCCCCGCCCTTCCACAACATCCCCAACATTGCCAACCTCC
GAGACGCCGCTCTCTTCCCCCTCCAGACGGCCACAGGACCACTCAAACCCTCCCTCCTCTTCCG
CTCCGCCGACGTCTCCAAATTGCTTCCCGAGAACTGGTCATCCCTCTCCGCCCTCGGCGTCACG
CACGTGTTCGACCTGCGCAGCGCCCCAGAAGTCGGCTTCACCTCATCGACCCCTTCCACCTCGC
TGCCTACCTGGGTATCGTCCATGAACGAAGCAGGCATCAACCGGACCTGGGTCCCCGTCTTCGC
GGAACAAGACTACTCGCCCGAAGGCCTGGCGAAGCGATATGTAAAGTACATGGACGAGTCGGTC
ACGGGCTTCGTCTCTGCTTACCACGACATCCTGCTCGCTGCGGGCCCTGCCTATCGCTCCATCC
TACTGTACCTGATCCGCTCCCCGGGCGCCGGTGTATTGGTGCACTGCACCGCCGGCAAAGACCG
GACGGGCATCTTCTTCGGCATCGTATTCGACTACCTGGGCGTCGATCGGCAGGCCATTGCCGAC
GAGTACAATCTGACCGAACAGGGACTGGGGAGCGTGCGGGAGGAGGTCGTAGCGAGGTTGATGA
AGAGCCCTGCGTTCCGCAACTATATGAACACGAAGCAGACGGGCGAGCAGCTTAGTACCGAGGA
GATTGGGCGCTTGATTCAAGAAGAGAAGGAGGGTAAGGCTCCGGCTGCTGAAGAAGAGCTCGAC
CCAGAGACGCGCGAGGTGGGGCGTCAGGCTGCGTTGAGGATGGTGGGCGCACGCAAGGAGACGA
TGATTGCGGCGCTGCAGATGGTGGATAAGGTGTTTGGGGGCTCGGAGAAGTATCTGAGGGAGTA
TTGTGGACTTGGGGATAAGGATCTCGAGGCGCTGAGGAGGAACCTGGTGGATGGGGCATGA
SEQ ID NO: 8; Pp. TPP optimized cDNA
ATGTCCAGTCCACAGGCGCTTCCTAGTCCACCCTTCCACAACATTCCAAACATCGCTAATTTGA
GAGACGCCGCTTTATTCCCATTACAAACTGCTACTGGTCCATTGAAGCCATCCTTATTGTTCCG
TTCTGCCGATGTCTCTAAATTGTTACCAGAAAACTGGTCCTCTTTGTCTGCTTTGGGTGTCACT
CACGTTTTCGATTTGAGATCTGCCCCAGAAGTTGGTTTCACTTCTTCTACCCCTTCTACCTCTT
TGCCAACTTGGGTTTCTTCCATGAACGAAGCTGGTATCAACAGAACTTGGGTCCCTGTTTTTGC
CGAACAAGACTACTCTCCAGAAGGTTTGGCTAAGAGATACGTCAAGTACATGGATGAATCTGTC
ACCGGTTTCGTCTCTGCCTATCATGATATTTTGTTGGCTGCTGGTCCTGCTTACAGATCTATCT
TGTTGTACTTGATCAGATCCCCAGGTGCCGGTGTTTTAGTTCACTGTACTGCCGGTAAAGATAG
AACTGGTATTTTCTTCGGTATTGTCTTCGATTACTTGGGTGTCGATCGTCAAGCCATTGCCGAC
GAATACAACTTGACTGAACAAGGTTTGGGTTCTGTCAGAGAAGAAGTCGTTGCCAGATTGATGA
AATCCCCTGCTTTCAGAAACTACATGAACACTAAGCAAACTGGTGAACAATTGTCTACTGAAGA
AATCGGTAGATTGATTCAAGAAGAAAAGGAAGGTAAAGCTCCAGCCGCCGAAGAAGAATTGGAC
CCAGAAACCAGAGAAGTTGGTCGTCAAGCTGCTTTGAGAATGGTTGGTGCTAGAAAAGAAACCA
TGATCGCCGCCTTGCAAATGGTCGACAAGGTTTTTGGTGGTTCTGAAAAGTACTTAAGAGAATA
CTGTGGTTTGGGTGATAAGGACTTGGAAGCTTTAAGAAGAAACTTAGTTGATGGTGCCTAA
SEQ ID NO: 9; Pp. TPP amino acid sequence
MSSPQALPSPPFHNIPNIANLRDAALFPLQTATGPLKPSLLFRSADVSKLLPENWSSLSALGVT
HVFDLRSAPEVGFISSIPSTSLPTWVSSMNEAGINRTWVPVFAEQDYSPEGLAKRYVKYMDESV
TGFVSAYHDILLAAGPAYRSILLYLIRSPGAGVLVHCTAGKDRTGIFFGIVEDYLGVDRQAIAD
EYNLTEQGLGSVREEVVARLMKSPAFRNYMNTKQTGEQLSTEEIGRLIQEEKEGKAPAAEEELD
PETREVGRQAALRMVGARKETMIAALQMVDKVFGGSEKYLREYCGLGDKDLEALRRNLVDGA
SEQ ID NO: 10; Ta. TPP wild-type cDNA
ATGTCTGATGACACCTTTCCCACGGCTGCTCCCGGGACAGTACCTTCTTCGCGGTTTCTTTCCG
TGGGCGGAGTAGTCAATTTCCGTGAACTGGGCGGTTACCCTTGCAGTGCTCTCCCTCCTGCCTC
AAACGGCTCACCGGACAACGCGTCGGATTCGACACCTCGGGGTAGCCACTCGTGCATCCGGCCT
GGATTTCTCTTTCGATCGGCTCAGCCGTCCCAGGTTACCCCAGCCGGTATCGAGACACTAGTAC
ACGAACTTCGCATCCGGGCGATTTTTGACTTTCGCTCACAGACCGAAATTCAGCTTGTCACCAC
TCGCTATCCTGATTCGCTACTCGAGATACCTGGTACGACTCGCTATTCCGTGCCGGTCTTCTCG
GAGGGTGATTATTCCCCGGCGTCATTAGTCAAGAAGTACGGAGTGTCCTCCGATACTGCCGTGG
ATTCCATTTCCTCCACAAACGCCAAGCCTACAGGATTCGTCCACGCGTATGAGGCCATCGCACG
CAGTGCTGCAGGAAACGGTAGTTTTCGTAAGATAACGGACCACATAATACAACATCCGGACCGG
CCAATCCTATTTCACTGCACACTGGGAAAAGACCGAACCGGTGTATTTGCAGCATTGCTATTGA
GTCTTTGCGGGGTACCAAACGAGACTATAGTTGAAGACTATGCTATGACTACTGAGGGCTTCGG
GGCCTGGCGAGAACACCTAGTCCAACGCTTGTTGCAAAGAAAGGATGCAGCTACACGTGAAGAG
GCAGAATCCATTATTGCCAGCCACCCGGAGACTATGAAGGCTTTTCTTGAAGATGTGGTAGCGG
CGAAGTTTGGGAGTGCTCGAAATTATTTTGTCCAGCAATGTGGATTAACGCAAGCAGACGTTGA
TAAATTAATTCATACATTGGTCTTTACGAATTGA
SEQ ID NO: 11; Ta. TPP optimized cDNA
ATGTCCGATGACACATTCCCGACCGCTGCTCCTGGTACTGTCCCATCCTCCAGATTCTTGTCCG
TCGGTGGTGTCGTCAACTTCAGAGAATTAGGTGGTTACCCTTGTTCTGCTTTGCCACCAGCCTC
CAATGGTTCCCCTGACAACGCTTCTGACTCTACCCCTAGAGGTTCCCATTCCTGTATCAGACCA
GGTTTCTTATTCCGTTCTGCTCAACCATCCCAAGTCACTCCAGCTGGTATCGAAACCTTGGTCC
ATGAATTGCGTATCCGTGCCATTTTTGACTTTAGATCTCAAACCGAGATTCAATTGGTCACTAC
TAGATACCCAGATTCCTTGTTAGAAATTCCAGGTACTACTAGATACTCTGTCCCAGTCTTCTCT
GAAGGTGACTACTCTCCAGCTTCTTTGGTCAAGAAATACGGTGTTTCTTCTGACACTGCTGTTG
ATTCTATTTCCTCTACCAATGCTAAACCTACCGGTTTCGTTCACGCTTACGAAGCCATTGCCAG
ATCCGCTGCTGGTAATGGTTCTTTTAGAAAGATTACCGACCACATTATTCAACACCCTGATAGA
CCTATTTTGTTTCACTGCACTTTAGGTAAGGATAGAACCGGTGTCTTCGCCGCTTTATTGTTGT
CTTTGTGTGGTGTCCCTAATGAAACTATTGTTGAAGACTACGCCATGACTACTGAAGGTTTCGG
TGCTTGGAGAGAACACTTGGTTCAAAGATTGTTGCAAAGAAAAGACGCTGCTACTAGAGAAGAA
GCTGAATCTATCATCGCCTCTCATCCAGAAACTATGAAAGCTTTCTTAGAGGATGTTGTTGCTG
CTAAGTTCGGTTCCGCTAGAAACTACTTCGTCCAACAATGTGGTTTGACTCAAGCTGACGTTGA
CAAGTTGATCCATACTTTGGTTTTCACCAACTAA
SEQ ID NO: 12; Ta. TPP amino acid sequence
MSDDTFPTAAPGTVPSSRFLSVGGVVNFRELGGYPCSALPPASNGSPDNASDSTPRGSHSCIRP
GFLFRSAQPSQVTPAGIETLVHELRIRAIFDFRSQTEIQLVTTRYPDSLLEIPGTTRYSVPVFS
EGDYSPASLVKKYGVSSDTAVDSISSTNAKPTGFVHAYEAIARSAAGNGSFRKITDHIIQHPDR
PILFHCTLGKDRTGVFAALLLSLCGVPNETIVEDYAMTTEGFGAWREHLVQRLLORKDAATREE
AESIIASHPETMKAFLEDVVAAKFGSARNYFVQQCGLTQADVDKLIHTLVFTN
SEQ ID NO: 13; Ss. TPP wild-type cDNA
ATGTCAACCTCAAACCCCCTCCCCTCCCCACCTTTCCACAACGTCCCCAACATCGCCAACCTCC
GAGACGCCGCTCTCTTCCCGCTCTCCACACCCTCTGGACCCCTCAAACCGTCTCTCCTCTTCCG
CTCCGCCGACGTCTCCAAGCTGCTTCCCGAGAACTGGTCCTCGCTCTCTAGCCTAGGTGTCACG
CACGTGTTCGACCTGCGCTCTGTGCCCGAAGTTGGCTTTTCCTCATCTACGCCCTCCGCCTCTT
TGCCAGCCTGGGTGTCGTCCATGCAGGATGCGGGGATCAACAGGACGTGGGTCCCCGTCTTCGC
GGAGGCGGACTATTCGCCTGAGGGACTTGCGAAGCGGTATGTGAAGTATATGGATGAGGCGGTG
GAGGGATTCGTGTCTGCATATCATGACATTCTTCTCTCTGCGGGCCCGGCGTATCGCGCTATCT
TGCTCTATCTGATCGATTCGCCCAACAAGGGCGTGTTGGTGCACTGCACAGCCGGCAAAGATCG
TACGGGCATCTTCTTCGGCGTCCTATTCGACTACCTCGGCGTTGACCGCCAAATCATCGCAGAT
GAATACAACCTCACTGAACTCGGCCTGGGCAGCGTGCGGGAAGAGGTCGTGGCGCGCTTACTCA
AGTCCGTTGCGTTCCGCAATTATATGAACACGAAGCAGACAGGCAAAGAACTGAGCACGGAGGA
AATTGGACGTTTGATTCAGGAGGAGAAGGAGGGCAAGACGCCAGATGTGGAGGAGGAGCTGAGT
CCCGAGACGCGCGAGGTGGGGCGCCAGGCTGCGCTGCGCATGGTGGGGGCGCGCAAGGAGACGA
TGCTTAAGGCGCTCGAGATGGTGGATAGTGAGTTTGGTGGCGCGGAGAAGTATCTGAGGGAGTA
TTGTGGGCTGGGCGATGAGGAGCTGGAGAAGCTGAGGAAGAATCTGGTGCAGGGCGCGTGA
SEQ ID NO: 14; Ss. TPP optimized cDNA
ATGTCAACTTCAAACCCCTTACCCAGCCCACCATTCCATAACGTCCCAAACATCGCTAACTTGA
GAGACGCCGCCTTGTTCCCATTGTCCACTCCTTCCGGTCCATTGAAGCCTTCTTTATTGTTCAG
ATCCGCTGACGTCTCTAAGTTGTTGCCAGAAAACTGGTCCTCTTTGTCTTCCTTGGGTGTTACT
CACGTTTTCGATTTGAGATCTGTTCCAGAAGTTGGTTTCTCTTCCTCTACTCCATCTGCCTCCT
TGCCAGCTTGGGTTTCTTCTATGCAAGACGCCGGTATTAACAGAACTTGGGTTCCTGTTTTCGC
TGAAGCCGATTACTCTCCAGAAGGTTTAGCTAAGAGATACGTTAAATACATGGACGAAGCTGTC
GAAGGTTTCGTTTCTGCTTACCACGATATTTTGTTGTCCGCTGGTCCAGCTTACAGAGCTATCT
TGTTGTACTTGATTGATTCTCCAAACAAGGGTGTCTTGGTTCACTGCACTGCTGGTAAGGACAG
AACCGGTATCTTTTTCGGTGTCTTGTTTGATTACTTGGGTGTTGACAGACAAATCATTGCCGAC
GAGTACAACTTGACTGAATTGGGTTTAGGTTCTGTTAGAGAAGAAGTTGTTGCCAGATTGTTGA
AGTCCGTTGCCTTCAGAAACTACATGAACACTAAACAAACTGGTAAGGAATTGTCTACTGAGGA
AATTGGTAGATTGATTCAAGAAGAAAAGGAAGGTAAGACTCCAGACGTCGAAGAAGAATTGTCT
CCAGAAACCAGAGAAGTTGGTAGACAAGCTGCTTTAAGAATGGTTGGTGCTAGAAAAGAAACTA
TGTTGAAGGCCTTGGAAATGGTCGACTCTGAGTTTGGTGGTGCTGAAAAGTACTTGAGAGAATA
CTGCGGTTTGGGTGACGAAGAATTGGAAAAGTTGAGAAAGAACTTAGTCCAAGGTGCTTAA
SEQ ID NO: 15; Ss. TPP amino acid sequence
MSTSNPLPSPPFHNVPNIANLRDAALFPLSTPSGPLKPSLLERSADVSKLLPENWSSLSSLGVT
HVFDLRSVPEVGFSSSTPSASLPAWVSSMQDAGINRTWVPVFAEADYSPEGLAKRYVKYMDEAV
EGFVSAYHDILLSAGPAYRAILLYLIDSPNKGVLVHCTAGKDRTGIFFGVLFDYLGVDRQIIAD
EYNLTELGLGSVREEVVARLLKSVAFRNYMNTKQTGKELSTEEIGRLIQEEKEGKTPDVEEELS
PETREVGRQAALRMVGARKETMLKALEMVDSEFGGAEKYLREYCGLGDEELEKLRKNLVQGA
SEQ ID NO: 16; Ap. TPP wild-type cDNA
ATGGCTGAACTATTCGACAAGGCACCCGAACTGCCCCCACCATTCGTAAATGTAACAGGCATCG
CCAACTTCCGCGACATTGGCGGCTACAGCACCACCGACAAGCACTCAATCCGCCGAGGTCTCGT
CTTCCGCGCCGCCGACCCGAGCAAAGTGCAACCAGAAGGCCTTGCCAAATTGAAAGAGCTAGGA
GTGAAGAAGGTATTCGATCTTCGCAGTATACCCGAGATCCACAAGCAAGGTCCGGAATGGCAGG
GTGTTGAAGTTGAGAAGGAGGTGTTCATCACCAAGTCCGAAGGTAGTGTTGAAGCTGTCCCAGA
AGGAGATATTGAAAGGATATGGTGTCCGGTATTCCGAAACACGGATTATGGTCCTGAGCAAGTC
GCCATCAGGTTCCAGAATTATGCTAAGAAGGGGAGCGAGGGATTCGTAAAAGCGTACGCAGATA
TCATGGCGAATGGCCCTACTGCATACAGATCCATCCTCACCCATCTCGCTCAACCAAACCCTTC
GCCATGTATAATCCACTGCACGGCAGGCAAAGACCGCACCGGCGTCATCTGCGCGATCCTGTAT
CTCCTCTGCTCCGTCCCCGCCACCACAGTAGCAAAAGAATACTCCCTCACCGACACCGGACTAC
AGCATTTAGTCCCTCTATTCACAGAACGCCTGCTCAAGAACCCAGCATTAGTTGGCAACGAGGA
AGGAGTCAAGAATATGATTTCCGCACAGCCGGAGAAGATGGCTGCGACTATTGAGATGATGAAG
GAGGTTTATGGTGGGGCGGAGGGGTATGTGAGGGAGGTTGTTGGATTGAGTGGGGAGCAAATTG
AGCAGATCAGGAAGAATCTTTTGAGTGACGAGGAGGCTGTCTTTTGA
SEQ ID NO: 17; Ap. TPP optimized cDNA
ATGGCAGAATTATTCGACAAGGCACCTGAATTGCCACCTCCTTTCGTCAATGTTACTGGTATTG
CTAACTTCCGTGACATCGGTGGTTACTCTACTACTGACAAGCACTCTATTCGTAGAGGTTTGGT
CTTCAGAGCTGCTGACCCATCTAAAGTTCAACCTGAAGGTTTGGCTAAGTTGAAGGAATTGGGT
GTCAAGAAGGTCTTCGATTTGAGATCCATCCCAGAAATCCACAAGCAAGGTCCAGAATGGCAAG
GTGTCGAAGTTGAAAAGGAAGTTTTCATCACTAAGTCTGAAGGTTCCGTCGAAGCCGTTCCAGA
AGGTGATATTGAAAGAATCTGGTGTCCTGTTTTCCGTAATACCGACTATGGTCCAGAACAAGTT
GCTATTAGATTCCAAAACTACGCTAAGAAAGGTTCCGAAGGTTTTGTTAAAGCCTATGCCGACA
TCATGGCCAACGGTCCAACTGCTTACAGATCTATCTTGACCCATTTGGCTCAACCTAATCCATC
TCCTTGTATCATTCACTGTACTGCTGGTAAGGATAGAACTGGTGTTATCTGCGCTATTTTGTAC
TTGTTATGTTCCGTTCCAGCTACCACTGTTGCTAAGGAATATTCTTTGACTGACACTGGTTTAC
AACACTTGGTTCCATTATTTACCGAAAGATTGTTGAAGAACCCTGCCTTGGTTGGTAACGAAGA
AGGTGTTAAGAACATGATCTCCGCTCAACCAGAAAAGATGGCTGCTACTATTGAAATGATGAAG
GAAGTCTATGGTGGTGCTGAGGGTTACGTCAGAGAAGTTGTCGGTTTGTCTGGTGAACAAATCG
AACAAATTCGTAAGAACTTGTTGTCCGACGAAGAAGCCGTTTTCTAA
SEQ ID NO: 18; Ap. TPP amino acid sequence
MAELFDKAPELPPPFVNVTGIANFRDIGGYSTTDKHSIRRGLVFRAADPSKVQPEGLAKLKELG
VKKVFDLRSIPEIHKQGPEWQGVEVEKEVFITKSEGSVEAVPEGDIERIWCPVFRNTDYGPEQV
AIRFQNYAKKGSEGFVKAYADIMANGPTAYRSILTHLAQPNPSPCIIHCTAGKDRIGVICAILY
LLCSVPATTVAKEYSLTDTGLQHLVPLFTERLLKNPALVGNEEGVKNMISAQPEKMAATIEMMK
EVYGGAEGYVREVVGLSGEQIEQIRKNLLSDEEAVF
SEQ ID NO: 19; TalVeTPP wild-type cDNA
ATGTCTAATGACACCACTACCACGGCTTCTGCCGGAACAGCAACTTCTTCGCGGTTTCTTTCCG
TGGGGGGAGTTGTGAACTTCCGTGAACTGGGCGGTTACCCATGTGATTCTGTCCCTCCTGCTCC
TGCCTCAAACGGCTCACCGGACAATGCATCTGAAGCGACCCTTTGGGTTGGCCACTCGTCCATT
CGGCCTGGATTTCTGTTTCGATCGGCACAGCCGTCTCAGATTACCCCGGCCGGTATTGAGACAT
TGATCCGCCAGCTTGGCATCCAGACAATTTTTGACTTTCGTTCAAGGACGGAAATTGAGCTTGT
TGCCACTCGCTATCCTGATTCGCTACTTGAGATACCTGGCACGACTCGCTATTCCGTGCCCGTC
TTCTCGGAAGGCGACTATTCCCCAGCGTCATTAGTCAAGAGGTACGGAGTGTCCTCCGATACTG
CAACCGATTCCACTTCCTCCAAAAGTGCTAAGCCTACAGGATTCGTCCACGCATATGAGGCTAT
CGCACGCAGTGCAGCAGAAAACGGCAGTTTTCGTAAGATAACGGACCACATAATACAACATCCG
GACCGGCCTATTCTGTTTCACTGTACACTGGGGAAAGACCGAACCGGTGTGTTTGCAGCATTGT
TATTGAGTCTTTGCGGGGTACCAGACGAGACGATAGTTGAAGACTATGCTATGACTACCGAGGG
ATTTGGAGCCTGGCGGGAACATCTAATTCAACGCTTGCTACAAAGGAAGGATGCAGCTACGCGC
GAGGATGCAGAATCCATTATTGCCAGCCCCCCGGAGACTATGAAGGCTTTTCTAGAAGATGTGG
TAGCAGCCAAGTTCGGGGGTGCTCGAAATTACTTTATCCAGCACTGTGGATTTACGGAAGCTGA
GGTTGATAAGTTAAGCCATACACTGGCCATTACGAATTGA
SEQ ID NO: 20; TalVeTPP optimized cDNA
ATGTCCAATGATACTACGACAACTGCTTCCGCCGGTACTGCTACTTCCTCCAGATTCTTGTCCG
TCGGTGGTGTTGTTAATTTCAGAGAATTAGGTGGTTACCCTTGTGATTCTGTTCCACCAGCTCC
AGCCTCCAATGGTTCCCCTGACAACGCTTCTGAGGCTACTTTGTGGGTTGGTCACTCTTCCATT
AGACCAGGTTTTTTGTTTAGATCCGCTCAACCTTCTCAAATCACTCCAGCCGGTATTGAAACTT
TGATCAGACAATTGGGTATTCAAACTATCTTCGATTTCAGATCTAGAACTGAAATTGAATTGGT
TGCTACTAGATACCCAGATTCTTTGTTAGAAATTCCAGGTACCACTCGTTACTCCGTCCCAGTC
TTCTCCGAAGGTGACTATTCCCCAGCTTCTTTGGTTAAAAGATACGGTGTTTCTTCTGACACCG
CCACTGATTCCACTTCCTCTAAGTCCGCTAAACCTACCGGTTTCGTTCACGCTTATGAAGCCAT
CGCCAGATCCGCCGCTGAAAACGGTTCTTTCCGTAAGATCACCGACCATATCATTCAACACCCA
GACAGACCAATTTTGTTTCATTGTACTTTGGGTAAGGATAGAACTGGTGTCTTCGCTGCTTTAT
TGTTGTCTTTATGTGGTGTCCCTGATGAAACTATTGTTGAAGATTACGCCATGACTACTGAAGG
TTTTGGTGCTTGGAGAGAACACTTAATCCAAAGATTGTTGCAAAGAAAGGATGCTGCTACTAGA
GAAGACGCTGAATCTATCATTGCTTCCCCACCAGAAACTATGAAGGCTTTCTTGGAAGATGTTG
TTGCTGCTAAGTTTGGTGGTGCTAGAAACTACTTCATCCAACACTGCGGTTTCACTGAAGCTGA
AGTCGACAAGTTGTCTCATACTTTGGCTATTACTAACTAA
SEQ ID NO: 21; TalVeTPP amino acid sequence
MSNDTTTTASAGTATSSRFLSVGGVVNFRELGGYPCDSVPPAPASNGSPDNASEATLWVGHSSI
RPGFLERSAQPSQITPAGIETLIRQLGIQTIFDERSRTEIELVATRYPDSLLEIPGTTRYSVPV
FSEGDYSPASLVKRYGVSSDTATDSTSSKSAKPTGFVHAYEAIARSAAENGSFRKITDHIIQHP
DRPILFHCTLGKDRTGVFAALLLSLCGVPDETIVEDYAMTTEGFGAWREHLIQRLLQRKDAATR
EDAESIIASPPETMKAFLEDVVAAKFGGARNYFIQHCGFTEAEVDKLSHTLAITN
SEQ ID NO: 22; PvCPS wild-type cDNA
ATGAGCCCAATGGATTTACAAGAATCAGCGGCAGCTTTGGTGCGGCAGTTGGGGGAGAGAGTCG
AAGATCGCCGTGGTTTTGGATTCATGAGCCCTGCCATCTATGATACCGCATGGGTCTCTATGAT
TAGCAAGACAATCGATGACCAAAAAACATGGTTGTTTGCAGAATGTTTCCAGTACATTCTTTCT
CATCAGCTCGAAGACGGTGGTTGGGCAATGTATGCATCTGAAATCGACGCCATCCTAAACACTT
CGGCCTCATTACTATCATTAAAGAGACATCTTTCAAATCCCTATCAAATTACATCTATCACACA
AGAGGATCTGTCCGCCCGCATTAACAGGGCTCAGAATGCTTTACAGAAGCTTCTCAATGAGTGG
AATGTCGACAGCACGCTCCACGTGGGATTCGAGATCCTAGTTCCGGCCCTACTCAGGTATCTCG
AAGATGAGGGCATCGCTTTTGCTTTTTCTGGTAGAGAGCGCCTGCTTGAGATTGAGAAACAGAA
ATTATCAAAGTTCAAAGCACAGTATCTATACCTTCCAATCAAAGTGACAGCTTTGCATTCTCTG
GAAGCGTTCATAGGCGCCATTGAGTTTGATAAAGTCAGTCACCACAAAGTCAGCGGTGCGTTCA
TGGCATCTCCATCATCCACAGCAGCTTACATGATGCATGCGACACAATGGGATGATGAATGCGA
GGATTACCTACGCCACGTCATTGCTCATGCATCTGGGAAAGGATCCGGAGGTGTTCCAAGCGCT
TTTCCTTCCACCATCTTTGAAAGCGTTTGGCCTCTATCAACTCTGCTAAAGGTGGGATATGATC
TCAACTCGGCACCTTTTATCGAAAAAATCAGATCATACTTGCATGATGCATATATTGCTGAAAA
GGGAATTCTCGGCTTCACTCCTTTTGTTGGCGCTGATGCAGATGATACCGCTACCACCATATTG
GTGCTCAATCTTTTGAACCAACCAGTCTCAGTCGACGCGATGTTGAAGGAATTTGAAGAAGAAC
ATCACTTCAAAACCTACTCTCAGGAGCGCAATCCTAGTTTCTCGGCCAATTGTAACGTTCTTCT
TGCCTTACTATACAGTCAAGAGCCATCGCTTTATAGCGCGCAGATCGAAAAAGCTATAAGGTTC
CTCTATAAGCAATTCACAGATTCAGAAATGGACGTTCGAGACAAATGGAATCTATCACCATACT
ATTCTTGGATGCTCATGACACAAGCCATCACGCGGTTGACGACTCTTCAGAAGACTTCGAAACT
TTCAACATTGAGAGATGATTCTATCAGCAAAGGCTTGATTAGTCTGCTGTTTAGGATAGCTTCT
ACCGTGGTTAAAGACCAAAAGCCAGGAGGTTCTTGGGGCACTCGAGCTTCGAAAGAAGAGACTG
CCTACGCAGTGTTGATTCTCACATATGCTTTCTACCTCGATGAGGTTACGGAGTCGTTGCGGCA
TGATATCAAGATCGCCATTGAGAATGGTTGCTCATTCCTATCTGAAAGAACCATGCAGTCCGAT
TCGGAGTGGCTTTGGGTTGAGAAAGTCACATATAAATCAGAGGTTCTTTCGGAAGCATATATCT
TGGCCGCTCTTAAACGGGCAGCTGACTTACCCGACGAAAATGCAGAAGCAGCCCCCGTCATAAA
TGGAATTTCTACAAATGGATTTGAGCATACCGATAGAATTAACGGCAAGCTTAAAGTCAATGGT
ACCAACGGTACAAATGGCAGTCATGAGACAAACGGTATCAACGGTACGCATGAAATTGAACAGA
TCAATGGCGTCAACGGCACGAATGGTCACTCTGATGTGCCTCACGATACAAATGGCTGGGTAGA
AGAGCCGACCGCCATCAATGAGACAAATGGCCACTACGTGAATGGCACGAATCACGAGACTCCC
CTTACCAACGGCATTTCCAATGGAGATTCTGTTTCCGTTCATACAGACCACTCGGACAGTTACT
ATCAGCGCAGTGATTGGACAGCCGACGAAGAACAAATTCTTCTCGGTCCATTTGACTACCTGGA
GAGCCTGCCAGGCAAGAATATGCGCTCACAACTGATTCAATCATTCAACACATGGCTCAAAGTC
CCAACTGAGAGCTTGGATGTTATTATTAAGGTGATTTCAATGTTGCATACGGCCTCTCTCTTGA
TCGATGATATTCAGGATCAATCAATACTCCGCCGCGGGCAACCTGTAGCGCACAGCATCTTTGG
CACAGCGCAAGCAATGAACTCAGGGAATTATGTCTACTTTCTAGCCCTTAGGGAGGTTCAGAAA
CTACAAAACCCGAAAGCCATCAGTATTTATGTTGACTCTTTGATTGATCTTCACCGTGGCCAAG
GCATGGAGCTTTTCTGGCGGGATTCTCTCATGTGCCCAACCGAAGAGCAGTACCTTGACATGGT
CGCAAACAAAACTGGCGGCCTGTTTTGCCTTGCTATCCAATTGATGCAAGCTGAAGCCACTATC
CAAGTCGACTTCATACCACTTGTCCGACTACTCGGCATCATCTTCCAGATTTGTGATGATTACT
TGAATCTGAAGTCTACGGCCTATACAGACAACAAAGGGTTGTGTGAGGATTTGACAGAGGGCAA
ATTCTCTTTTCCTATCATCCATAGCATTCGATCCAACCCTGGCAACCGACAGCTAATCAACATC
TTGAAGCAAAAGCCACGTGAAGACGACATCAAACGCTATGCTCTATCCTATATGGAAAGCACCA
ACTCATTTGAGTATACTCGGGGTGTCGTTAGAAAACTGAAGACCGAGGCAATCGATACTATTCA
AGGCTTGGAGAAGCACGGCCTGGAAGAGAATATTGGCATTCGAAAGATACTAGCTCGCATGTCC
CTTGAGCTATGA
SEQ ID NO: 23; PvCPS optimized cDNA
ATGTCACCGATGGACCTTCAGGAGAGTGCCGCTGCTTTGGTCCGTCAATTAGGTGAAAGAGTCG
AAGATCGTAGAGGTTTCGGTTTTATGTCCCCAGCCATTTATGACACTGCCTGGGTTTCTATGAT
TTCCAAGACCATTGATGATCAAAAGACTTGGTTGTTCGCCGAATGCTTTCAATACATTTTGTCT
CACCAATTAGAAGACGGTGGTTGGGCTATGTACGCCTCCGAAATCGATGCTATTTTGAACACCT
CTGCCTCTTTGTTGTCCTTGAAAAGACACTTATCTAACCCATACCAAATTACTTCTATCACTCA
AGAAGATTTGTCTGCTAGAATCAACAGAGCTCAAAACGCTTTGCAAAAGTTGTTGAACGAGTGG
AACGTTGATTCTACCTTGCATGTTGGTTTCGAAATCTTAGTTCCAGCCTTGTTGAGATACTTAG
AAGATGAAGGTATTGCTTTCGCCTTCTCTGGTAGAGAAAGATTGTTGGAAATCGAAAAGCAAAA
GTTGTCTAAGTTCAAAGCTCAATACTTGTACTTACCAATTAAGGTCACCGCTTTACATTCCTTG
GAAGCTTTCATTGGTGCCATCGAATTTGACAAGGTTTCTCATCATAAGGTTTCCGGTGCTTTCA
TGGCTTCTCCATCCTCTACTGCTGCTTATATGATGCACGCCACTCAATGGGATGATGAATGTGA
GGACTACTTAAGACACGTCATTGCCCATGCTTCTGGTAAAGGTTCTGGTGGTGTCCCTTCTGCT
TTCCCATCCACCATCTTTGAATCTGTTTGGCCATTATCTACCTTGTTAAAAGTCGGTTATGATT
TGAACTCTGCTCCATTCATCGAAAAGATCAGATCTTACTTGCACGACGCCTACATTGCTGAAAA
AGGTATCTTAGGTTTTACTCCATTTGTTGGTGCCGATGCTGACGACACCGCTACTACTATCTTG
GTTTTGAACTTGTTGAACCAACCTGTCTCCGTTGATGCTATGTTGAAAGAATTCGAAGAGGAGC
ATCACTTTAAGACCTATTCTCAAGAACGTAACCCATCTTTCTCCGCTAACTGTAACGTTTTGTT
GGCTTTGTTGTACTCCCAAGAGCCATCCTTATATTCTGCTCAAATTGAAAAGGCCATTCGTTTC
TTGTACAAACAATTCACTGACTCTGAAATGGACGTTAGAGATAAGTGGAACTTGTCTCCATACT
ACTCTTGGATGTTGATGACCCAAGCCATCACCCGTTTAACTACCTTACAAAAGACTTCCAAATT
GTCCACCTTGAGAGATGACTCCATTTCTAAGGGTTTGATCTCTTTGTTATTCCGTATCGCTTCT
ACTGTTGTCAAGGACCAAAAACCAGGTGGTTCTTGGGGTACTAGAGCCTCCAAAGAAGAAACTG
CTTACGCCGTTTTGATCTTGACTTACGCTTTTTACTTAGACGAAGTTACCGAATCTTTGCGTCA
TGACATCAAGATTGCCATTGAAAACGGTTGCTCTTTCTTGTCTGAGAGAACTATGCAATCTGAC
TCCGAATGGTTGTGGGTCGAGAAGGTCACTTACAAATCCGAAGTCTTGTCCGAAGCTTACATTT
TGGCTGCCTTAAAGAGAGCTGCCGATTTGCCAGATGAAAATGCTGAAGCTGCTCCAGTTATTAA
TGGTATCTCTACTAACGGTTTCGAACACACTGATAGAATTAACGGTAAGTTGAAGGTTAACGGT
ACTAACGGTACCAACGGTTCCCATGAAACTAACGGTATTAACGGTACCCACGAAATTGAACAAA
TCAACGGTGTCAACGGTACTAATGGTCATTCTGATGTTCCACACGATACTAACGGTTGGGTCGA
GGAACCAACTGCTATCAACGAAACTAACGGTCACTATGTTAACGGTACCAATCACGAAACTCCA
TTAACCAACGGTATTTCTAATGGTGACTCTGTTTCCGTTCATACTGACCACTCTGACTCTTATT
ATCAACGTTCTGATTGGACTGCTGACGAAGAACAAATCTTGTTAGGTCCATTTGACTACTTGGA
ATCTTTGCCAGGTAAAAACATGCGTTCTCAATTGATCCAATCCTTCAATACCTGGTTGAAGGTC
CCAACTGAATCTTTGGACGTCATCATCAAGGTTATTTCTATGTTGCATACCGCCTCCTTATTGA
TTGATGATATTCAAGACCAATCCATCTTGCGTCGTGGTCAACCTGTCGCTCACTCTATCTTCGG
TACTGCTCAAGCCATGAATTCTGGTAACTACGTCTACTTCTTAGCTTTAAGAGAAGTTCAAAAG
TTGCAAAACCCAAAGGCTATTTCTATTTACGTCGACTCTTTGATTGACTTGCACAGAGGTCAAG
GTATGGAATTGTTCTGGAGAGATTCTTTAATGTGTCCTACTGAAGAACAATACTTGGATATGGT
TGCTAACAAGACCGGTGGTTTGTTCTGCTTGGCCATTCAATTGATGCAAGCTGAAGCCACTATT
CAAGTCGACTTCATTCCATTGGTCAGATTGTTGGGTATCATCTTCCAAATTTGTGACGATTACT
TGAACTTGAAGTCTACTGCCTACACCGATAACAAGGGTTTGTGTGAAGATTTGACTGAAGGTAA
GTTTTCTTTCCCAATCATCCACTCTATTAGATCTAACCCAGGTAACCGTCAATTGATCAACATC
TTGAAGCAAAAACCAAGAGAAGACGACATTAAGAGATACGCCTTGTCTTACATGGAATCCACCA
ACTCTTTCGAATACACTAGAGGTGTTGTTAGAAAATTGAAGACCGAAGCTATCGATACCATCCA
AGGTTTAGAAAAGCACGGTTTGGAGGAGAATATTGGTATCCGTAAGATTTTGGCCCGTATGTCC
TTGGAATTGTAA
SEQ ID NO: 24; PvCPS amino acid sequence
MSPMDLQESAAALVRQLGERVEDRRGFGEMSPAIYDTAWVSMISKTIDDQKTWLFAECFQYILS
HQLEDGGWAMYASEIDAILNTSASLLSLKRHLSNPYQITSITQEDLSARINRAQNALQKLLNEW
NVDSTLHVGFEILVPALLRYLEDEGIAFAFSGRERLLEIEKQKLSKFKAQYLYLPIKVTALHSL
EAFIGAIEFDKVSHHKVSGAFMASPSSTAAYMMHATQWDDECEDYLRHVIAHASGKGSGGVPSA
FPSTIFESVWPLSTLLKVGYDLNSAPFIEKIRSYLHDAYIAEKGILGFTPFVGADADDTATTIL
VLNLLNQPVSVDAMLKEFEEEHHFKTYSQERNPSFSANCNVLLALLYSQEPSLYSAQIEKAIRE
LYKQFTDSEMDVRDKWNLSPYYSWMLMTQAITRLITLQKTSKLSTLRDDSISKGLISLLFRIAS
TVVKDQKPGGSWGTRASKEETAYAVLILTYAFYLDEVTESLRHDIKIAIENGCSFLSERTMQSD
SEWLWVEKVTYKSEVLSEAYILAALKRAADLPDENAEAAPVINGISTNGFEHTDRINGKLKVNG
TNGINGSHETNGINGTHEIEQINGVNGINGHSDVPHDINGWVEEPTAINETNGHYVNGINHETP
LINGISNGDSVSVHTDHSDSYYQRSDWTADEEQILLGPFDYLESLPGKNMRSQLIQSENTWLKV
PTESLDVIIKVISMLHTASLLIDDIQDQSILRRGQPVAHSIFGTAQAMNSGNYVYFLALREVQK
LQNPKAISIYVDSLIDLHRGQGMELFWRDSLMCPTEEQYLDMVANKTGGLFCLAIQLMQAEATI
QVDFIPLVRLLGIIFQICDDYLNLKSTAYTDNKGLCEDLTEGKFSFPIIHSIRSNPGNRQLINI
LKQKPREDDIKRYALSYMESTNSFEYTRGVVRKLKTEAIDTIQGLEKHGLEENIGIRKILARMS
LEL
SEQ ID NO: 25; pGAL1
TGGAACTTTCAGTAATACGCTTAACTGCTCATTGCTATATTGAAGTACGGATTAGAAGCCGCCG
AGCGGGCGACAGCCCTCCGACGGAAGACTCTCCTCCGTGCGTCCTGGTCTTCACCGGTCGCGTT
CCTGAAACGCAGATGTGCCTCGCGCCGCACTGCTCCGAACAATAAAGATTCTACAATACTAGCT
TTTATGGTTATGAAGAGGAAAAATTGGCAGTAACCTGGCCCCACAAACCTTCAAATCAACGAAT
CAAATTAACAACCATAGGATAATAATGCGATTAGTTTTTTAGCCTTATTTCTGGGGTAATTAAT
CAGCGAAGCGATGATTTTTGATCTATTAACAGATATATAAATGCAAAAGCTGCATAACCACTTT
AACTAATACTTTCAACATTTTCGGTTTGTATTACTTCTTATTCAAATGTCATAAAAGTATCAAC
AAAAAATTGTTAATATACCTCTATACTTTAACGTCAAGGAGAAAAAACTATA
SEQ ID NO: 26; pGAL10
CATCGCTTCGCTGATTAATTACCCCAGAAATAAGGCTAAAAAACTAATCGCATTATTATCCTAT
GGTTGTTAATTTGATTCGTTGATTTGAAGGTTTGTGGGGCCAGGTTACTGCCAATTTTTCCTCT
TCATAACCATAAAAGCTAGTATTGTAGAATCTTTATTGTTCGGAGCAGTGCGGCGCGAGGCACA
TCTGCGTTTCAGGAACGCGACCGGTGAAGACCAGGACGCACGGAGGAGAGTCTTCCGTCGGAGG
GCTGTCGCCCGCTCGGCGGCTTCTAATCCGTACTTCAATATAGCAATGAGCAGTTAAGCGTATT
ACTGAAAGTTCCAAAGAGAAGGTTTTTTTAGGCTAAGATAATGGGGCTCTTTACATTTCCACAA
CATATAAGTAAGATTAGATATGGATATGTATATGGTGGTATTGCCATGTAATATGATTATTAAA
CTTCTTTGCGTCCATCCAAAAAAAAAGTAAGAATTTTTGAAAATTCAATATAA
SEQ ID NO: 27; pGAL2
GGCTTAAGTAGGTTGCAATTTCTTTTTCTATTAGTAGCTAAAAATGGGTCACGTGATCTATATT
CGAAAGGGGCGGTTGCCTCAGGAAGGCACCGGCGGTCTTTCGTCCGTGCGGAGATATCTGCGCC
GTTCAGGGGTCCATGTGCCTTGGACGATATTAAGGCAGAAGGCAGTATCGGGGCGGATCACTCC
GAACCGAGATTAGTTAAGCCCTTCCCATCTCAAGATGGGGAGCAAATGGCATTATACTCCTGCT
AGAAAGTTAACTGTGCACATATTCTTAAATTATACAATGTTCTGGAGAGCTATTGTTTAAAAAA
CAAACATTTCGCAGGCTAAAATGTGGAGATAGGATTAGTTTTGTAGACATATATAAACAATCAG
TAATTGGATTGAAAATTTGGTGTTGTGAATTGCTCTTCATTATGCACCTTATTCAATTATCATC
AAGAATAGCAATAGTTAAGTAAACACAAGATTAACATAATAAAAAAAATAATTCTTTCATA
SEQ ID NO: 28; pGAL3
TTTTACTATTATCTTCTACGCTGACAGTAATATCAAACAGTGACACATATTAAACACAGTGGTT
TCTTTGCATAAACACCATCAGCCTCAAGTCGTCAAGTAAAGATTTCGTGTTCATGCAGATAGAT
AACAATCTATATGTTGATAATTAGCGTTGCCTCATCAATGCGAGATCCGTTTAACCGGACCCTA
GTGCACTTACCCCACGTTCGGTCCACTGTGTGCCGAACATGCTCCTTCACTATTTTAACATGTG
GAATTCTTGAAAGAATGAAATCGCCATGCCAAGCCATCACACGGTCTTTTATGCAATTGATTGA
CCGCCTGCAACACATAGGCAGTAAAATTTTTACTGAAACGTATATAATCATCATAAGCGACAAG
TGAGGCAACACCTTTGTTACCACATTGACAACCCCAGGTATTCATACTTCCTATTAGCGGAATC
AGGAGTGCAAAAAGAGAAAATAAAAGTAAAAAGGTAGGGCAACACATAGT
SEQ ID NO: 29; pGAL7
GGACGGTAGCAACAAGAATATAGCACGAGCCGCGAAGTTCATTTCGTTACTTTTGATATCGCTC
ACAACTATTGCGAAGCGCTTCAGTGAAAAAATCATAAGGAAAAGTTGTAAATATTATTGGTAGT
ATTCGTTTGGTAAAGTAGAGGGGGTAATTTTTCCCCTTTATTTTGTTCATACATTCTTAAATTG
CTTTGCCTCTCCTTTTGGAAAGCTATACTTCGGAGCACTGTTGAGCGAAGGCTCATTAGATATA
TTTTCTGTCATTTTCCTTAACCCAAAAATAAGGGAAAGGGTCCAAAAAGCGCTCGGACAACTGT
TGACCGTGATCCGAAGGACTGGCTATACAGTGTTCACAAAATAGCCAAGCTGAAAATAATGTGT
AGCTATGTTCAGTTAGTTTGGCTAGCAAAGATATAAAAGCAGGTCGGAAATATTTATGGGCATT
ATTATGCAGAGCATCAACATGATAAAAAAAAACAGTTGAATATTCCCTCAAAA
SEQ ID NO: 30; pGAL4
GCGACACAGAGATGACAGACGGTGGCGCAGGATCCGGTTTAAACGAGGATCCCTTAAGTTTAAA
CAACAACAGCAAGCAGGTGTGCAAGACACTAGAGACTCCTAACATGATGTATGCCAATAAAACA
CAAGAGATAAACAACATTGCATGGAGGCCCCAGAGGGGCGATTGGTTTGGGTGCGTGAGCGGCA
AGAAGTTTCAAAACGTCCGCGTCCTTTGAGACAGCATTCGCCCAGTATTTTTTTTATTCTACAA
ACCTTCTATAATTTCAAAGTATTTACATAATTCTGTATCAGTTTAATCACCATAATATCGTTTT
CTTTGTTTAGTGCAATTAATTTTTCCTATTGTTACTTCGGGCCTTTTTCTGTTTTATGAGCTAT
TTTTTCCGTCATCCTTCCCCAGATTTTCAGCTTCATCTCCAGATTGTGTCTACGTAATGCACGC
CATCATTTTAAGAGAGGACAGAGAAGCAAGCCTCCTGAAAG
SEQ ID NO: 31; pMAL1
GATGATGGACACTAGTGTGTCGAGAATGTATCAACTATATATAGTCCTAATGCCACACAAATAT
GAAGTGGGGGAAGCCCATTCTTAATCCGGCTCAATTTTGGTGCGTGATCGCGGCCTATGTTTGC
TTCCAGAAAAAGCTTAGAATAATATTTCTCACCTTTGATGGAATGCTCGCGAGTGCTCGTTTTG
ATTACCCCATATGCATTGTTGCAGCATGCAAGCACTATTGCAAGCCACGCATGGAAGAAATTTG
CAAACACCTATAGCCCCGCGTTGTTGAGGAGGTGGACTTGGTGTAGGACCATAAAGCTGTGCAC
TACTATGGTGAGCTCTGTCGTCTGGTGACCTTCTATCTCAGGCACATCCTCGTTTTTGTGCATG
AGGTTCGAGTCACGCCCACGGCCTATTAATCCGCGAAATAAATGCGAAATCTAAATTATGACGC
AAGGCTGAGAGATTCTGACACGCCGCATTTGCGGGGCAGTAATTATCGGGCAGTTTTCCGGGGT
TCGGGATGGGGTTTGGAGAGAAAGTTCAACACAGACCAAAACAGCTTGGGACCACTTGGATGGA
GGTCCCCGCAGAAGAGCTCTGGCGCGTTGGACAAACATTGACAATCCACGGCAAAATTGTCTAC
AGTTCCGTGTATGCGGATAGGGATATCTTCGGGAGTATCGCAATAGGATACAGGCACTGTGCAG
ATTACGCGACATGATAGCTTTGTATGTTCTACAGACTCTGCCGTAGCAGTCTAGATATAATATC
GGAGTTTTGTAGCGTCGTAAGGAAAACTTGGGTTACACAGGTTTCTTGAGAGCCCTTTGACGTT
GATTGCTCTGGCTTCCATCCAGGCCCTCATGTGGTTCAGGTGCCTCCGCAGTGGCTGGCAAGCG
TGGGGGTCAATTACGTCACTTCTATTCATGTACCCCAGACTCAATTGTTGACAGCAATTTCAGC
GAGAATTAAATTCCACAATCAATTCTCGCTGAAATAATTAGGCCGTGATTTAATTCTCGCTGAA
ACAGAATCCTGTCTGGGGTACAGATAACAATCAAGTAACTATTATGGACGTGCATAGGAGGTGG
AGTCCATGACGCAAAGGGAAATATTCATTTTATCCTCGCGAAGTTGGGATGTGTCAAAGCGTCG
CGCTCGCTATAGTGATGAGAATGTCTTTAGTAAGCTTAAGCCATATAAAGACCTTCCGCCTCCA
TATTTTTTTTTATCCCTCTTGACAATATTAATTCCTT
SEQ ID NO: 32; pMAL2
AAGGAATTAATATTGTCAAGAGGGATAAAAAAAAATATGGAGGCGGAAGGTCTTTATATGGCTT
AAGCTTACTAAAGACATTCTCATCACTATAGCGAGCGCGACGCTTTGACACATCCCAACTTCGC
GAGGATAAAATGAATATTTCCCTTTGCGTCATGGACTCCACCTCCTATGCACGTCCATAATAGT
TACTTGATTGTTATCTGTACCCCAGACAGGATTCTGTTTCAGCGAGAATTAAATCACGGCCTAA
TTATTTCAGCGAGAATTGATTGTGGAATTTAATTCTCGCTGAAATTGCTGTCAACAATTGAGTC
TGGGGTACATGAATAGAAGTGACGTAATTGACCCCCACGCTTGCCAGCCACTGCGGAGGCACCT
GAACCACATGAGGGCCTGGATGGAAGCCAGAGCAATCAACGTCAAAGGGCTCTCAAGAAACCTG
TGTAACCCAAGTTTTCCTTACGACGCTACAAAACTCCGATATTATATCTAGACTGCTACGGCAG
AGTCTGTAGAACATACAAAGCTATCATGTCGCGTAATCTGCACAGTGCCTGTATCCTATTGCGA
TACTCCCGAAGATATCCCTATCCGCATACACGGAACTGTAGACAATTTTGCCGTGGATTGTCAA
TGTTTGTCCAACGCGCCAGAGCTCTTCTGCGGGGACCTCCATCCAAGTGGTCCCAAGCTGTTTT
GGTCTGTGTTGAACTTTCTCTCCAAACCCCATCCCGAACCCCGGAAAACTGCCCGATAATTACT
GCCCCGCAAATGCGGCGTGTCAGAATCTCTCAGCCTTGCGTCATAATTTAGATTTCGCATTTAT
TTCGCGGATTAATAGGCCGTGGGCGTGACTCGAACCTCATGCACAAAAACGAGGATGTGCCTGA
GATAGAAGGTCACCAGACGACAGAGCTCACCATAGTAGTGCACAGCTTTATGGTCCTACACCAA
GTCCACCTCCTCAACAACGCGGGGCTATAGGTGTTTGCAAATTTCTTCCATGCGTGGCTTGCAA
TAGTGCTTGCATGCTGCAACAATGCATATGGGGTAATCAAAACGAGCACTCGCGAGCATTCCAT
CAAAGGTGAGAAATATTATTCTAAGCTTTTTCTGGAAGCAAACATAGGCCGCGATCACGCACCA
AAATTGAGCCGGATTAAGAATGGGCTTCCCCCACTTCATATTTGTGTGGCATTAGGACTATATA
TAGTTGATACATTCTCGACACACTAGTGTCCATCATC
SEQ ID NO: 33; pMAL11
GCGCCTCAAGAAAATGATGCTGCAAGAAGAATTGAGGAAGGAACTATTCATCTTACGTTGTTTG
TATCATCCCACGATCCAAATCATGTTACCTACGTTAGGTACGCTAGGAACTAAAAAAAGAAAAG
AAAAGTATGCGTTATCACTCTTCGAGCCAATTCTTAATTGTGTGGGGTCCGCGAAAATTTCCGG
ATAAATCCTGTAAACTTTAACTTAAACCCCGTGTTTAGCGAAATTTTCAACGAAGCGCGCAATA
AGGAGAAATATTATCTAAAAGCGAGAGTTTAAGCGAGTTGCAAGAATCTCTACGGTACAGATGC
AACTTACTATAGCCAAGGTCTATTCGTATTACTATGGCAGCGAAAGGAGCTTTAAGGTTTTAAT
TACCCCATAGCCATAGATTCTACTCGGTCTATCTATCATGTAACACTCCGTTGATGCGTACTAG
AAAATGACAACGTACCGGGCTTGAGGGACATACAGAGACAATTACAGTAATCAAGAGTGTACCC
AACTTTAACGAACTCAGTAAAAAATAAGGAATGTCGACATCTTAATTTTTTATATAAAGCGGTT
TGGTATTGATTGTTTGAAGAATTTTCGGGTTGGTGTTTCTTTCTGATGCTACATAGAAGAACAT
CAAACAACTAAAAAAATAGTATAAT
SEQ ID NO: 34; pMAL12
ATTATACTATTTTTTTAGTTGTTTGATGTTCTTCTATGTAGCATCAGAAAGAAACACCAACCCG
AAAATTCTTCAAACAATCAATACCAAACCGCTTTATATAAAAAATTAAGATGTCGACATTCCTT
ATTTTTTACTGAGTTCGTTAAAGTTGGGTACACTCTTGATTACTGTAATTGTCTCTGTATGTCC
CTCAAGCCCGGTACGTTGTCATTTTCTAGTACGCATCAACGGAGTGTTACATGATAGATAGACC
GAGTAGAATCTATGGCTATGGGGTAATTAAAACCTTAAAGCTCCTTTCGCTGCCATAGTAATAC
GAATAGACCTTGGCTATAGTAAGTTGCATCTGTACCGTAGAGATTCTTGCAACTCGCTTAAACT
CTCGCTTTTAGATAATATTTCTCCTTATTGCGCGCTTCGTTGAAAATTTCGCTAAACACGGGGT
TTAAGTTAAAGTTTACAGGATTTATCCGGAAATTTTCGCGGACCCCACACAATTAAGAATTGGC
TCGAAGAGTGATAACGCATACTTTTCTTTTCTTTTTTTAGTTCCTAGCGTACCTAACGTAGGTA
ACATGATTTGGATCGTGGGATGATACAAACAACGTAAGATGAATAGTTCCTTCCTCAATTCTTC
TTGCAGCATCATTTTCTTGAGGCGCTCTGGGCAAGGTATAAAAAGTTCCATTAATACGTCTCTA
AAAAATTAAATCATCCATCTCTTAAGCAGTTTTTTTGATAATCTCAAATGTACATCAGTCAAGC
GTAACTAAATTACATAA
SEQ ID NO: 35; pMAL31
TTATGTATTTTAGTTACGCTTGACTGATGTACATTTGAGATTATCAAAAAAACTGCTTAAGAGA
TAGATGGTTTAATTTTTTAGAGACGTATTAATGGAACTTTTTATACCTTGCCCAGAGCGCCTCA
AGAAAATGATGCTGAAAGAAGAATTGAGGAAGGAACTACTCATCTTACGTTGTTTGTATCATCC
CACGATCCAAATCATGTTACCTACGTTAGGTACGCTAGGAACTGAAAAAAGAAAAGAAAAGTAT
GCGTTATCACTCTTCGAGCCAATTCTTAATTGTGTGGGGTCCGCGAAAACTTCCGGATAAATCC
TGTAAACTTAAACTTAAACCCCGTGTTTAGCGAAATTTTCAACGAAGCGCGCAATAAGGAGAAA
TATTATATAAAAGCGAGAGTTTAAGCGAGGTTGCAAGAATCTCTACGGTACAGATGCAACTTAC
TATAGCCAAGGTCTATTCGTATTGGTATCCAAGCAGTGAAGCTACTCAGGGGAAAACATATTTT
CAGAGATCAAAGTTATGTCAGTCTCTTTTTCATGTGTAACTTAACGTTTGTGCAGGTATCATAC
CGGCCTCCACATAATTTTTGTGGGGAAGACGTTGTTGTAGCAGTCTCCTTATACTCTCCAACAG
GTGTTTAAAGACTTCTTCAGGCCTCATAGTCTACATCTGGAGACAACATTAGATAGAAGTTTCC
ACAGAGGCAGCTTTCAATATACTTTCGGCTGTGTACATTTCATCCTGAGTGAGCGCATATTGCA
TAAGTACTCAGTATATAAAGAGACACAATATACTCCATACTTGTTGTGAGTGGTTTTAGCGTAT
TCAGTATAACAATAAGAATTACATCCAAGACTATTAATTAACT
SEQ ID NO: 36; pMAL32
AGTTAATTAATAGTCTTGGATGTAATTCTTATTGTTATACTGAATACGCTAAAACCACTCACAA
CAAGTATGGAGTATATTGTGTCTCTTTATATACTGAGTACTTATGCAATATGCGCTCACTCAGG
ATGAAATGTACACAGCCGAAAGTATATTGAAAGCTGCCTCTGTGGAAACTTCTATCTAATGTTG
TCTCCAGATGTAGACTATGAGGCCTGAAGAAGTCTTTAAACACCTGTTGGAGAGTATAAGGAGA
CTGCTACAACAACGTCTTCCCCACAAAAATTATGTGGAGGCCGGTATGATACCTGCACAAACGT
TAAGTTACACATGAAAAAGAGACTGACATAACTTTGATCTCTGAAAATATGTTTTCCCCTGAGT
AGCTTCACTGCTTGGATACCAATACGAATAGACCTTGGCTATAGTAAGTTGCATCTGTACCGTA
GAGATTCTTGCAACCTCGCTTAAACTCTCGCTTTTATATAATATTTCTCCTTATTGCGCGCTTC
GTTGAAAATTTCGCTAAACACGGGGTTTAAGTTTAAGTTTACAGGATTTATCCGGAAGTTTTCG
CGGACCCCACACAATTAAGAATTGGCTCGAAGAGTGATAACGCATACTTTTCTTTTCTTTTTTC
AGTTCCTAGCGTACCTAACGTAGGTAACATGATTTGGATCGTGGGATGATACAAACAACGTAAG
ATGAGTAGTTCCTTCCTCAATTCTTCTTTCAGCATCATTTTCTTGAGGCGCTCTGGGCAAGGTA
TAAAAAGTTCCATTAATACGTCTCTAAAAAATTAAACCATCTATCTCTTAAGCAGTTTTTTTGA
TAATCTCAAATGTACATCAGTCAAGCGTAACTAAAATACATAA
SEQ ID NO: 37; Bt. GPPS wild-type cDNA
ATGTTGACCTCTAGCAAATCAATTGAATCCTTCCCCAAGAATGTTCAACCTTATGGCAAGCATT
ATCAAAATGGCTTGGAACCTGTTGGAAAAAGCCAAGAAGATATTCTCTTGGAGCCATTCCACTA
TCTCTGTTCGAATCCTGGTAAAGATGTCCGAACCAAGATGATTGAAGCGTTCAATGCTTGGCTG
AAAGTACCCAAGGACGATTTGATCGTCATCACACGTGTGATTGAAATGCTTCATAGTGCTAGTT
TGTTAATTGATGATGTGGAAGATGATTCCGTGTTGCGTCGTGGTGTTCCTGCAGCTCATCATAT
ATATGGTACTCCTCAAACTATCAATTGTGCTAATTACGTGTACTTTCTTGCACTGAAAGAAATT
GCCAAGTTGAACAAGCCCAACATGATTACTATCTATACCGATGAATTGATCAATTTGCACAGAG
GGCAAGGAATGGAATTGTTTTGGCGTGACACCTTAACTTGTCCTACAGAGAAAGAATTTCTTGA
CATGGTAAACGACAAAACTGGTGGCCTCTTGAGATTAGCTGTGAAACTTATGCAAGAAGCTAGT
CAATCGGGAACTGATTATACGGGACTCGTAAGTAAGATTGGTATCCATTTCCAAGTACGCGACG
ATTATATGAATTTGCAGTCAAAAAACTATGCTGACAACAAAGGATTCTGCGAAGACTTGACAGA
AGGAAAATTCTCTTTCCCTATTATACATTCAATCCGCTCTGACCCAAGCAATCGCCAGCTTTTG
AACATTTTAAAACAGCGCAGTAGCTCTATCGAACTCAAGCAATTTGCCTTGCAGCTACTGGAAA
ACACAAACACTTTCCAATACTGTCGTGATTTCTTACGTGTCTTGGAAAAGGAAGCTAGAGAAGA
AATTAAGCTTTTAGGGGGTAACATCATGTTGGAGAAAATTATGGATGTCTTGAGTGTCAATGAA
TAA
SEQ ID NO: 38; Bt. GPPS optimized cDNA
ATGTTGACATCTTCTAAGTCCATCGAATCTTTCCCAAAGAACGTTCAACCATACGGTAAACACT
ATCAAAACGGTTTAGAACCAGTCGGTAAGTCTCAAGAAGACATCTTGTTGGAACCTTTCCACTA
CTTATGTTCTAATCCAGGTAAGGATGTTAGAACCAAGATGATTGAAGCTTTCAACGCCTGGTTG
AAAGTCCCAAAGGACGATTTGATTGTTATCACCAGAGTCATTGAAATGTTGCACTCCGCTTCTT
TGTTGATTGATGACGTCGAGGACGATTCTGTCTTGAGAAGAGGTGTCCCAGCCGCCCACCATAT
CTACGGTACCCCTCAAACCATCAACTGCGCTAACTACGTTTATTTCTTGGCCTTGAAAGAAATC
GCCAAGTTGAACAAGCCAAATATGATTACTATTTATACCGATGAATTGATCAACTTGCACAGAG
GTCAAGGTATGGAATTGTTCTGGCGTGATACCTTGACCTGCCCAACTGAGAAAGAGTTTTTGGA
TATGGTTAACGATAAGACTGGTGGTTTGTTGAGATTGGCCGTCAAGTTGATGCAAGAGGCTTCT
CAATCTGGTACCGACTATACTGGTTTGGTTTCTAAGATCGGTATCCATTTTCAAGTTAGAGATG
ACTACATGAACTTGCAATCCAAAAACTACGCCGATAATAAGGGTTTCTGTGAAGATTTGACCGA
AGGTAAGTTCTCCTTTCCAATTATTCACTCTATCAGATCTGACCCATCCAACAGACAATTATTG
AATATTTTGAAGCAAAGATCTTCTTCTATTGAATTGAAACAATTCGCTTTACAATTGTTAGAAA
ACACTAACACTTTTCAATACTGTAGAGATTTCTTGAGAGTTTTGGAAAAGGAAGCCAGAGAAGA
GATCAAATTATTGGGTGGTAACATCATGTTGGAAAAGATTATGGACGTCTTGTCTGTTAATGAA
TAA
SEQ ID NO: 39; Bt. GPPS amino acid sequence
MLTSSKSIESFPKNVQPYGKHYQNGLEPVGKSQEDILLEPFHYLCSNPGKDVRIKMIEAFNAWL
KVPKDDLIVITRVIEMLHSASLLIDDVEDDSVLRRGVPAAHHIYGTPQTINCANYVYFLALKEI
AKLNKPNMITIYTDELINLHRGQGMELFWRDTLTCPTEKEFLDMVNDKTGGLLRLAVKLMQEAS
QSGTDYTGLVSKIGIHFQVRDDYMNLQSKNYADNKGFCEDLTEGKFSFPIIHSIRSDPSNRQLL
NILKORSSSIELKQFALQLLENTNTFQYCRDFLRVLEKEAREEIKLLGGNIMLEKIMDVLSVNE
SEQ ID NO: 40; Cf. CPPS wild-type cDNA
ATGTCATGGATGAACAACGGTAAAAACCTTAACTGCCAACTTACTCACAAGAAAATATCGAAAG
TAGCCGAGATTCGAGTTGCCACGGTGAACGCGCCGCCGGTGCACGATCAAGACGATTCCACAGA
AAATCAGTGCCATGACGCGGTGAATAATATTGAGGATCCGATCGAATACATAAGAACGCTGCTG
AGGACGACAGGGGACGGCCGAATAAGTGTGTCGCCGTATGACACTGCGTGGGTCGCTCTGATCA
AGGACTTGCAAGGACGCGATGCCCCCGAGTTTCCGTCGAGCCTGGAGTGGATCATACAGAATCA
GCTGGCCGATGGGTCGTGGGGCGATGCCAAGTTCTTCTGTGTGTATGATCGCCTCGTGAATACG
ATAGCATGCGTGGTGGCCTTGAGATCATGGGATGTTCATGCTGAAAAGGTGGAAAGAGGAGTGA
GATACATCAATGAAAATGTGGAAAAGCTTAGAGATGGAAATGAGGAACACATGACTTGIGGGTT
CGAAGTGGTGTTTCCTGCGCTTCTGCAGAGAGCTAAGAGCTTAGGGATCCAAGATCTTCCCTAT
GATGCTCCCGTCATTCAAGAGATATATCACTCCAGGGAACAAAAGTTGAAAAGGATTCCACTGG
AGATGATGCACAAAGTGCCAACTTCTTTATTATTTAGTCTGGAAGGGCTGGAGAATTTGGAGTG
GGATAAGCTTTTGAAACTGCAGTCAGCTGATGGCTCTTTCCTCACTTCTCCCTCCTCCACTGCC
TTCGCTTTTATGCAAACTCGTGATCCTAAATGCTACCAATTCATCAAAAACACTATTCAAACTT
TCAACGGAGGAGCACCACACACTTATCCTGTCGATGTTTTTGGAAGACTTTGGGCAATCGACAG
GCTGCAGCGCCTCGGGATTTCTCGCTTCTTTGAGTCCGAGATTGCTGATTGCATCGCCCACATC
CACAGGTTTTGGACAGAGAAGGGAGTTTTCAGTGGAAGAGAATCAGAGTTTTGCGACATTGATG
ATACATCCATGGGAGTCCGACTCATGAGAATGCATGGATACGATGTTGATCCAAATGTATTGAA
GAACTTCAAAAAGGATGACAAGTTTTCATGCTACGGTGGACAGATGATTGAGTCTCCGTCTCCC
ATTTACAATCTCTACAGGGCTTCCCAACTCCGCTTCCCCGGTGAGCAAATTCTCGAAGATGCCA
ACAAATTTGCCTACGATTTCTTACAAGAAAAGCTTGCCCACAACCAGATTCTTGATAAATGGGT
TATATCTAAGCACTTGCCTGATGAGATAAAACTGGGACTGGAGATGCCGTGGTACGCCACCCTA
CCCCGCGTGGAGGCAAGATACTACATACAGTACTATGCTGGTTCAGGCGATGTATGGATCGGAA
AGACTCTCTACAGGATGCCCGAGATCAGCAACGATACATATCATGAGCTTGCAAAAACAGACTT
CAAGAGATGCCAAGCTCAGCATCAGTTTGAGTGGATTTACATGCAAGAATGGTACGAGAGTTGC
AACATGGAAGAATTCGGGATAAGCAGAAAGGAGCTTCTGGTTGCTTACTTCTTGGCGACTGCAA
GCATATTCGAGCTGGAGAGGGCTAATGAGAGAATCGCCTGGGCCAAATCCCAAATCATTTCCAC
CATCATTGCATCTTTCTTCAATAACCAAAACACTTCACCGGAGGATAAACTTGCATTTTTAACA
GATTTCAAAAATGGCAACTCCACAAACATGGCTCTGGTGACCCTCACTCAATTCCTAGAGGGAT
TCGACAGATACACTAGCCATCAGTTGAAGAATGCCTGGAGCGTATGGCTGAGAAAGCTGCAGCA
AGGAGAAGGCAACGGCGGCGCAGACGCAGAGCTCCTAGTAAACACATTGAACATTTGTGCCGGC
CACATTGCCTTTAGGGAAGAAATACTCGCACACAACGACTACAAGACTCTCTCCAACCTGACTA
GCAAAATCTGTCGACAACTTTCTCAAATTCAAAATGAAAAGGAGTTGGAGACAGAGGGACAGAA
AACAAGCATAAAAAACAAGGAACTGGAAGAAGATATGCAAAGACTGGTGAAGTTGGTGTTGGAG
AAATCAAGGGTTGGAATCAACAGAGATATGAAGAAAACATTTCTTGCAGTGGTAAAAACTTATT
ACTACAAAGCATATCATTCTGCTCAGGCCATCGACAACCATATGTTCAAAGTACTTTTCGAACC
AGTCGCCCTCGAGTGCTG
SEQ ID NO: 41; Sm. CPPS wild type cDNA
ATGGCCTCCTTATCCTCTACAATCCTCAGCCGCTCTCCGGCGGCCCGCCGCAGAATTACGCCGG
CGTCGGCTAAGCTTCACCGGCCGGAATGTTTCGCCACCAGTGCATGGATGGGCAGCAGCAGTAA
AAACCTTTCTCTCAGCTACCAACTTAATCACAAGAAAATATCAGTTGCCACAGTAGATGCGCCG
CAGGTGCATGACCACGACGGCACTACCGTTCATCAAGGCCATGATGCGGTGAAGAATATTGAGG
ATCCCATTGAATACATCAGGACGTTGTTGAGGACGACGGGGGACGGGAGAATAAGCGTGTCGCC
GTACGACACGGCGTGGGTGGCGATGATCAAGGACGTGGAGGGGGGGGACGGCCCCCAGTTCCCC
TCCAGCCTCGAGTGGATCGTGCAGAATCAACTCGAGGATGGATCGTGGGGCGATCAGAAGCTTT
TCTGCGTCTACGATCGCCTCGTCAATACCATCGCGTGCGTGGTAGCCTTGAGATCGTGGAATGT
TCATGCTCACAAGGTCAAAAGAGGAGTGACGTACATCAAGGAAAATGTGGATAAACTTATGGAG
GGAAATGAGGAGCACATGACTTGTGGGTTCGAAGTGGTGTTTCCGGCGCTTCTACAAAAAGCGA
AAAGCTTAGGCATCGAAGATCTTCCTTACGATTCTCCGGCGGTGCAGGAGGTTTATCATGTCAG
GGAACAAAAGTTGAAAAGGATTCCACTGGAGATTATGCACAAAATACCGACATCATTATTATTT
AGTTTGGAAGGGCTCGAAAATTTGGATTGGGACAAACTTTTGAAACTGCAGTCAGCCGACGGTT
CCTTCCTCACCTCTCCCTCCTCCACCGCCTTCGCGTTCATGCAAACCAAGGATGAAAAATGCTA
CCAATTCATCAAGAACACGATAGACACTTTCAACGGAGGAGCGCCACACACTTATCCCGTCGAC
GTGTTTGGAAGGCTCTGGGCGATCGACCGGCTGCAGCGCCTCGGAATTTCCCGCTTTTTTGAGC
CGGAGATTGCTGATTGCTTAAGCCACATCCACAAATTTTGGACGGATAAGGGAGTTTTCAGTGG
GAGAGAATCGGAGTTTTGCGACATTGACGATACATCCATGGGAATGAGGCTTATGAGGATGCAT
GGATATGATGTTGATCCAAATGTGCTGAGGAATTTCAAGCAGAAAGATGGTAAATTCTCTTGCT
ACGGCGGGCAGATGATCGAGTCGCCTTCTCCGATATACAATCTTTACAGAGCTTCTCAGCTCCG
ATTTCCCGGCGAGGAAATCCTCGAAGATGCGAAGAGATTCGCCTACGATTTCTTGAAAGAAAAA
CTAGCCAACAATCAGATTCTGGATAAATGGGTTATTTCTAAGCACTTGCCTGATGAGATCAAGC
TCGGGCTAGAGATGCCGTGGCTCGCCACCCTACCCCGCGTCGAGGCGAAGTACTACATCCAGTA
CTACGCCGGCTCCGGCGACGTGTGGATCGGAAAGACGCTGTACAGGATGCCGGAGATCAGCAAC
GACACGTACCACGACCTAGCCAAGACGGATTTCAAGAGATGCCAAGCGAAGCATCAGTTCGAGT
GGCTCTACATGCAAGAATGGTACGAGAGCTGCGGCATCGAGGAATTCGGGATAAGCAGAAAGGA
CCTTCTGCTTTCCTATTTCTTGGCGACCGCGAGCATCTTCGAGCTCGAGAGGACCAACGAGCGA
ATCGCGTGGGCCAAATCGCAGATCATCGCTAAGATGATCACTTCTTTCTTCAACAAGGAAACTA
CGTCGGAGGAGGACAAGCGAGCTCTTTTGAACGAGCTCGGAAACATTAATGGCCTCAACGACAC
AAACGGCGCAGGGAGAGAAGGTGGGGCCGGTAGCATTGCGCTAGCGACCCTCACTCAGTTCCTC
GAGGGATTCGACAGATACACCAGACACCAGCTGAAAAATGCTTGGAGCGTATGGCTGACGCAGC
TGCAACATGGCGAAGCAGACGACGCGGAGCTCCTAACCAACACGTTGAACATCTGCGCCGGCCA
CATCGCCTTCAGGGAAGAAATACTGGCGCACAACGAGTACAAAGCTCTCTCCAACCTAACCAGC
AAAATCTGTCGACAGCTTTCTTTCATTCAAAGCGAAAAGGAGATGGGAGTAGAGGGCGAGATCG
CAGCGAAATCGAGCATAAAAAACAAGGAACTCGAAGAAGACATGCAAATGTTGGTGAAGTTGGT
GCTTGAGAAATATGGGGGCATAGATAGAAATATAAAGAAAGCGTTTTTAGCAGTTGCGAAGACT
TATTATTACAGAGCGTATCATGCCGCCGACACCATAGACACACACATGTTTAAAGTGCTTTTCG
AGCCAGTCGCGTGA
SEQ ID NO: 42; Cf. CPPS amino acid sequence
MGSLSTMNLNHSPMSYSGILPSSSAKAKLLLPGCFSISAWMNNGKNLNCQLTHKKISKVAEIRV
ATVNAPPVHDQDDSTENQCHDAVNNIEDPIEYIRILLRITGDGRISVSPYDTAWVALIKDLQGR
DAPEFPSSLEWIIQNQLADGSWGDAKFFCVYDRLVNTIACVVALRSWDVHAEKVERGVRYINEN
VEKLRDGNEEHMTCGFEVVFPALLORAKSLGIQDLPYDAPVIQEIYHSREQKSKRIPLEMMHKV
PTSLLFSLEGLENLEWDKLLKLQSADGSFLTSPSSTAFAFMQTRDPKCYQFIKNTIQTENGGAP
HTYPVDVFGRLWAIDRLORLGISRFFESEIADCIAHIHRFWTEKGVFSGRESEFCDIDDTSMGV
RLMRMHGYDVDPNVLKNFKKDDKFSCYGGQMIESPSPIYNLYRASQLRFPGEQILEDANKFAYD
FLQEKLAHNQILDKWVISKHLPDEIKLGLEMPWYATLPRVEARYYIQYYAGSGDVWIGKTLYRM
PEISNDTYHELAKTDFKRCQAQHQFEWIYMQEWYESCNMEEFGISRKELLVAYFLATASIFELE
RANERIAWAKSQIISTIIASFFNNQNTSPEDKLAFLTDFKNGNSINMALVILTQFLEGFDRYTS
HQLKNAWSVWLRKLQQGEGNGGADAELLVNILNICAGHIAFREEILAHNDYKTLSNLTSKICRQ
LSQIQNEKELETEGQKISIKNKELEEDMQRLVKLVLEKSRVGINRDMKKTFLAVVKTYYYKAYH
SAQAIDNHMFKVLFEPVA
SEQ ID NO:43; Sm. CPPS amino acid sequence
MATVDAPQVHDHDGTTVHQGHDAVKNIEDPIEYIRTLLRTTGDGRISVSPYDTAWVAMIKDVEG
RDGPQFPSSLEWIVQNQLEDGSWGDQKLFCVYDRLVNTIACVVALRSWNVHAHKVKRGVTYIKE
NVDKLMEGNEEHMTCGFEVVFPALLQKAKSLGIEDLPYDSPAVQEVYHVREQKLKRIPLEIMHK
IPTSLLFSLEGLENLDWDKLLKLQSADGSFLTSPSSTAFAFMQTKDEKCYQFIKNTIDTENGGA
PHTYPVDVFGRLWAIDRLORLGISRFFEPEIADCLSHIHKFWTDKGVFSGRESEFCDIDDTSMG
MRLMRMHGYDVDPNVLRNFKQKDGKFSCYGGQMIESPSPIYNLYRASQLRFPGEEILEDAKRFA
YDFLKEKLANNQILDKWVISKHLPDEIKLGLEMPWLAILPRVEAKYYIQYYAGSGDVWIGKTLY
RMPEISNDTYHDLAKTDFKRCQAKHQFEWLYMQEWYESCGIEEFGISRKDLLLSYFLATASIFE
LERTNERIAWAKSQIIAKMITSFFNKETISEEDKRALLNELGNINGLNDINGAGREGGAGSIAL
ATLTQFLEGFDRYTRHQLKNAWSVWLTQLQHGEADDAELLINILNICAGHIAFREEILAHNEYK
ALSNLISKICRQLSFIQSEKEMGVEGEIAAKSSIKNKELEEDMQMLVKLVLEKYGGIDRNIKKA
FLAVAKTYYYRAYHAADTIDTHMFKVLFEPVA
SEQ ID NO: 44; ERG20 wild-type cDNA
ATGGCTTCAGAAAAAGAAATTAGGAGAGAGAGATTCTTGAACGTTTTCCCTAAATTAGTAGAGG
AATTGAACGCATCGCTTTTGGCTTACGGTATGCCTAAGGAAGCATGTGACTGGTATGCCCACTC
ATTGAACTACAACACTCCAGGCGGTAAGCTAAATAGAGGTTTGTCCGTTGTGGACACGTATGCT
ATTCTCTCCAACAAGACCGTTGAACAATTGGGGCAAGAAGAATACGAAAAGGTTGCCATTCTAG
GTTGGTGCATTGAGTTGTTGCAGGCTTACTTCTTGGTCGCCGATGATATGATGGACAAGTCCAT
TACCAGAAGAGGCCAACCATGTTGGTACAAGGTTCCTGAAGTTGGGGAAATTGCCATCAATGAC
GCATTCATGTTAGAGGCTGCTATCTACAAGCTTTTGAAATCTCACTTCAGAAACGAAAAATACT
ACATAGATATCACCGAATTGTTCCATGAGGTCACCTTCCAAACCGAATTGGGCCAATTGATGGA
CTTAATCACTGCACCTGAAGACAAAGTCGACTTGAGTAAGTTCTCCCTAAAGAAGCACTCCTTC
ATAGTTACTTTCAAGACTGCTTACTATTCTTTCTACTTGCCTGTCGCATTGGCCATGTACGTTG
CCGGTATCACGGATGAAAAGGATTTGAAACAAGCCAGAGATGTCTTGATTCCATTGGGTGAATA
CTTCCAAATTCAAGATGACTACTTAGACTGCTTCGGTACCCCAGAACAGATCGGTAAGATCGGT
ACAGATATCCAAGATAACAAATGTTCTTGGGTAATCAACAAGGCATTGGAACTTGCTTCCGCAG
AACAAAGAAAGACTTTAGACGAAAATTACGGTAAGAAGGACTCAGTCGCAGAAGCCAAATGCAA
AAAGATTTTCAATGACTTGAAAATTGAACAGCTATACCACGAATATGAAGAGTCTATTGCCAAG
GATTTGAAGGCCAAAATTTCTCAGGTCGATGAGTCTCGTGGCTTCAAAGCTGATGTCTTAACTG
CGTTCTTGAACAAAGTTTACAAGAGAAGCAAATAG
SEQ ID NO: 45; ERG20 amino acid sequence
MASEKEIRRERFLNVFPKLVEELNASLLAYGMPKEACDWYAHSLNYNTPGGKLNRGLSVVDTYA
ILSNKTVEQLGQEEYEKVAILGWCIELLQAYFLVADDMMDKSITRRGQPCWYKVPEVGEIAIND
AFMLEAAIYKLLKSHFRNEKYYIDITELFHEVTFQTELGQLMDLITAPEDKVDLSKFSLKKHSF
IVTFKTAYYSFYLPVALAMYVAGITDEKDLKQARDVLIPLGEYFQIQDDYLDCFGTPEQIGKIG
TDIQDNKCSWVINKALELASAEQRKILDENYGKKDSVAEAKCKKIFNDLKIEQLYHEYEESIAK
DLKAKISQVDESRGFKADVLTAFLNKVYKRSK

1. A genetically modified host cell capable of producing E-copalol, wherein the genetically modified host cell comprises one or more heterologous nucleic acids that each, independently, encodes an enzyme comprising an amino acid sequence that is at least 90% identical to the amino acid sequence of SEQ ID NOS. 3, 6, 9, 12, 15, or 18.

2. The genetically modified host cell of claim 1, wherein the enzyme comprises the amino acid sequence of SEQ ID NOS. 3, 6, 9, 12, 15, or 18.

3. A genetically modified host cell capable of producing E-copalol, wherein the genetically modified host cell comprises one or more heterologous nucleic acids that each, independently, encodes an enzyme capable of converting copalyl-diphosphate (CPP) to E-copalol.

4. The genetically modified host cell of claim 3, wherein the enzyme capable of converting CPP to E-copalol is a pyrophosphatase.

5. The genetically modified host cell of claim 1, further comprising one or more heterologous nucleic acids that each, independently, encodes one or more enzymes of a pathway for making E-copalol.

6. The genetically modified host cell of claim 1, further comprising one or more heterologous nucleic acids that each, independently, encodes an enzyme comprising the amino acid sequence of SEQ ID NO. 24, SEQ ID NO. 39, SEQ ID NO. 42, SEQ ID NO. 43, or SEQ ID NO. 45.

7. The genetically modified host cell of claim 1, further comprising one or more heterologous nucleic acids that each, independently, encodes an enzyme capable of converting one or more IPP, DMAPP, GPP, FPP, or GGPP into GPP, FPP, GGPP, or CPP.

8. The genetically modified host cell of claim 1, further comprising a CPP synthase, an Erg20, a GPP synthase, or a GGPP synthase.

9. The genetically modified host cell of claim 1, wherein the enzyme is under the control of a single transcriptional regulator.

10. The genetically modified host cell of any one of claims 1-8, wherein the enzyme is under the control of multiple transcriptional regulators.

11. The genetically modified host cell of claim 1, wherein the host cell is a yeast cell or a yeast strain.

12. The genetically modified host cell of claim 11, wherein the yeast cell or the yeast strain is Saccharomyces cerevisiae.

13. A fermentation composition, comprising:
a) the genetically modified host cell of claim 1;
b) optionally an overlay; and
c) E-copalol produced by the genetically modified host cell.

14. A method for producing E-copalol, comprising:
a) culturing the genetically modified host cell of claim 1 in a medium with a carbon source under conditions suitable for making E-copalol;
b) optionally providing an overlay; and
c) recovering E-copalol from the genetically modified host cell, the overlay, or the medium.

15. A non-naturally occurring enzyme capable of converting CPP to E-copalol comprising an amino acid sequence that is at least 90% identical to the amino acid sequence of SEQ ID NOS. 3, 6, 9, 12, 15, or 18.

16. The non-naturally occurring enzyme of claim 15, wherein the non-naturally occurring enzyme comprises the amino acid sequence of SEQ ID NOS. 3, 6, 9, 12, 15, or 18.
